(19) United States
(12) Patent Application Publication
(10) Pub. No.: US 2026/0284445 A1
(43) Pub. Date: Sep. 24, 2026

(54) SIMULTANEOUS MRI AND ULTRASOUND GUIDANCE FOR HISTOTRIPSY SYSTEMS AND METHODS

(71) Applicant: THE REGENTS OF THE UNIVERSITY OF MICHIGAN, Ann Arbor, MI (US)

(72) Inventors: Zhen XU, Ann Arbor, MI (US); Timothy L. HALL, Ann Arbor, MI (US); Douglas NOLL, Ann Arbor, MI (US); Jonathan R. SUKOVICH, Ann Arbor, MI (US)

(21) Appl. No.: 19/471,955

(22) PCT Filed: Apr. 3, 2024
(86) PCT No.: PCT/US2024/022881

§ 371 Date: Oct. 2, 2025

Related U.S. Application Data
(60) Provisional application No. 63/493,882, filed on Apr. 3, 2023.

(51) Int. Cl. A61N 7/02

(52) U.S. Cl. CPC A61N 7/02

(57) ABSTRACT

A histotripsy therapy system configured for the treatment of tissue is provided, which may include any number of features. Provided herein are systems and methods that provide efficacious non-invasive and minimally invasive therapeutic, diagnostic and research procedures. Systems and methods are provided for performing transcranial histotripsy therapy of brain tissue. Features include systems and methods for monitoring the treatment and for correcting aberrations resulting from the ultrasound signals passing through the skull.

### PRIORITY CLAIM

This patent application claims priority to U.S. provisional patent application No. 63/493,882, titled “SIMULTANEOUS MRI AND ULTRASOUND GUIDANCE FOR HISTOTRIPSY SYSTEMS AND METHODS,” and filed on Apr. 3, 2023, which is herein incorporated by reference in its entirety.

### STATEMENT AS TO FEDERALLY SPONSORED RESEARCH

This invention was made with Government support under Grant No. R01-EB028309 awarded by the National Institutes of Health. The Government has certain rights in the invention.

### INCORPORATION BY REFERENCE

All publications and patent applications mentioned in this specification are herein incorporated by reference to the same extent as if each individual publication or patent application was specifically and individually indicated to be incorporated by reference.

### FIELD

The present disclosure details novel high intensity therapeutic ultrasound (HITU) systems configured to produce acoustic cavitation, methods, devices and procedures for the minimally and non-invasive treatment of healthy, diseased and/or injured tissue. The acoustic cavitation systems and methods described herein, also referred to Histotripsy, may include transducers, drive electronics, positioning robotics, imaging systems, and integrated treatment planning and control software to provide comprehensive treatment and therapy for soft tissues in a patient.

### BACKGROUND

Histotripsy, or pulsed ultrasound cavitation therapy, is a technology where extremely short, intense bursts of acoustic energy induce controlled cavitation (microbubble formation) within the focal volume. The vigorous expansion and collapse of these microbubbles mechanically homogenizes cells and tissue structures within the focal volume. This is a very different end result than the coagulative necrosis characteristic of thermal ablation. To operate within a non-thermal, Histotripsy realm; it is necessary to deliver acoustic energy in the form of high amplitude acoustic pulses with low duty cycle.

Compared with conventional focused ultrasound technologies, Histotripsy has important advantages: 1) the destructive process at the focus is mechanical, not thermal; 2) cavitation appears bright on ultrasound imaging thereby confirming correct targeting and localization of treatment; 3) treated tissue generally, but not always, appears darker (more hypoechoic) on ultrasound imaging, so that the operator knows what has been treated; and 4) Histotripsy produces lesions in a controlled and precise manner. It is important to emphasize that unlike thermal ablative technologies such as microwave, radiofrequency, high-intensity focused ultrasound (HIFU) cryo or radiation, Histotripsy relies on the mechanical action of cavitation for tissue destruction and not on heat, cold or ionizing energy.

Approximately 256,000 new patients are diagnosed with primary brain tumor annually worldwide and many more are diagnosed with brain metastases. The first-line treatment is craniotomy-based surgical resection, a highly invasive surgery associated with morbidities secondary to surgical trauma as well as injury to the surrounding cerebral parenchyma. Radiation therapy and drug-based chemotherapeutics both have significant limitations for brain tumor treatment. There is an urgent, unmet clinical need for a noninvasive, safe, and effective treatment for brain tumors.

Transcranial focused ultrasound (FUS) thermal ablation can be applied for noninvasive brain treatment, but it can only be used to treat the center of the brain due to overheating of the skull, greatly limiting its use for brain tumor treatment. Microbubble-mediated transcranial FUS can temporarily open the blood brain barrier (BBB) for drug delivery. The treatment efficacy of FUS BBB opening (BBBO) is still limited with non-uniform drug delivery in human, and it would be impractical to rely entirely on FUS-BBBO to treat a sizable brain tumor.

Transcranial histotripsy uses microsecond ultrasound (US) pulses to generate mechanical ablation in the brain via cavitation. Using a very low duty cycle (<0.1%) to minimize skull heating, transcranial histotripsy has the potential to overcome the above limitations by noninvasively ablating a wide range of locations and volumes in the brain without overheating the skull. Transcranial histotripsy may be used to treat primary and metastatic brain tumors alone or combined with FUS BBBO drug delivery.

Two major challenges remain. 1) Treatment location profiles for skulls with significant density variation and large thicknesses is still limited due to high attenuation for these skulls and high-pressure hotspots in the skull when targeting locations near the skull surface. 2) MRI can provide guidance and monitoring for transcranial histotripsy, but it cannot be used to image cavitation near or on the skull surface or provide real-time monitoring at the frame rate (≥50 Hz) histotripsy is delivered, and the imaging quality is less than desired using suboptimal MRI coils.

There is a need for a new transcranial histotripsy system to expand the location profile for diverse patients and treatment monitoring with improved imaging quality at a high temporal resolution and capability to image skull surface cavitation.

### SUMMARY OF THE DISCLOSURE

An MRI compatible histotripsy system is provided, comprising: a transducer array comprising a plurality of transducer elements; transmission circuitry coupled to the transducer array and configured to transmit histotripsy pulses into a target tissue; a MRI system configured to generate MR images of a target tissue of the subject; and one or more coils placed between the MRI system and a subject, the one or more coils being configured to receive MRI signals from the MRI system to improve an image signal-to-noise-ratio (SNR) of the MR images of the MRI system.

In some aspects, the one or more coils are ultrasound translucent.

In other aspects, the transducer array comprises a transcranial transducer array and the one or more coils are positioned on a stretchable fabric.

In some aspects, the transducer array is configured to deliver histotripsy pulses into a focal location to generate less than 2 deg C. of heating at the focal location.

In one aspect, the histotripsy pulses are modified from conventional histotripsy pulses to generate the heating.

In some aspects, the MRI system is configured to generate MR thermometry images of the heating at the focal location.

In some aspects, the system includes one or more processors configured to determine if cavitation is being generated at a focus of the transducer array based on the MR thermometry images.

In other aspects, the one or more processors are configured to measure a distance between a center of a heating map in the MR thermometry images and the focus.

In one aspect, the system ins configured to identify an apparent diffusion coefficient (ADC) in the MR images to identify tissue changes in the targe tissue.

In some aspects, the transducer array is MR compatible.

A transcranial ultrasound treatment system is provided, comprising: a magnetic resonance (MR)-compatible histotripsy therapy array; a MR-compatible mechanical positioner with translation and rotation mobility configured to move the histotripsy therapy array; a stereotactic frame configured to rigidly fix a patient's head and facilitate aberration correction; an acoustic coupler acoustically coupled to the histotripsy therapy array to provide for ultrasound transmission from the histotripsy therapy array to the patient's head; and a support structure configured to connect the MR-conditional mechanical positioner with the histotripsy therapy array and to support the patient's neck.

In some aspects, the system includes a magnetic resonance imaging (MRI) system configured to generate MR-images of a target tissue.

In another aspect, the system includes one or more coils placed between the MRI system and the patient, the one or more coils being configured to receive MRI signals from the MRI system to improve an image signal-to-noise-ratio (SNR) of the MR images of the MRI system.

In some aspects, the one or more coils are ultrasound translucent.

In other aspects, the transducer array comprises a transcranial transducer array and the one or more coils are positioned on a stretchable fabric.

In one aspect, the transducer array is configured to deliver histotripsy pulses into a focal location to generate less than 2 deg C. of heating at the focal location.

In other aspects, the histotripsy pulses are modified from conventional histotripsy pulses to generate the heating.

In one aspect, the MRI system is configured to generate MR thermometry images of the heating at the focal location.

In another aspect, the system includes one or more processors configured to determine if cavitation is being generated at a focus of the transducer array based on the MR thermometry images.

In some aspects, the one or more processors are configured to measure a distance between a center of a heating map in the MR thermometry images and the focus.

In some aspects, the system is configured to identify an apparent diffusion coefficient (ADC) in the MR images to identify tissue changes in the targe tissue.

A method is provided comprising: delivering histotripsy pulses from a histotripsy array through a patient's skull into brain tissue; receiving transcranial ACE signals with the histotripsy array; and generating a cavitation map of cavitation detected near or on the skull surface.

In one aspect, the method includes overlaying the cavitation map on a MR image.

In some aspects, the method includes phase aligning the received transcranial ACE signals against inverse transmit delays.

In one aspect, the method includes evaluating if the timings of the received transcranial ACE signals are temporally co-aligned.

A histotripsy method is provided, comprising: obtaining medical images of a patient's skull and a target tissue volume; placing a geometric focus of a histotripsy transducer array on the target tissue volume; delivering one or more histotripsy pulses into the target tissue volume to generate less than 2 deg. C of heating in the target tissue volume;

obtaining MRI thermometry images of the target tissue volume; and determining if an actual focus of the histotripsy transducer array is positioned at the geometric focus based on the MRI thermometry images.

In some aspects, the medical images comprise computed tomography (CT) images.

In one aspect, the method includes identifying an aberration correction algorithm based on the medical images.

In other aspects, the method includes receiving acoustic cavitation emission (ACE) signals; and comprising identifying an aberration correction algorithm based on the ACE signals.

In some aspects, the method includes receiving acoustic cavitation emission (ACE) signals; and comprising identifying an aberration correction algorithm based on the ACE signals and the medical images.

In another aspect, the method includes mechanically or electronically steering the histotripsy transducer array to position the actual focus at the geometric focus.

### BRIEF DESCRIPTION OF THE DRAWINGS

The novel features of the invention are set forth with particularity in the claims that follow. A better understanding of the features and advantages of the present invention will be obtained by reference to the following detailed description that sets forth illustrative embodiments, in which the principles of the invention are utilized, and the accompanying drawings of which:

FIGS. 1A-1B illustrate an ultrasound imaging and therapy system.

FIG. 2 is a system diagram showing a histotripsy therapy system that includes a therapy cart, a fluidics cart, and a coupling system.

FIGS. 3A-3C show a transcranial histotripsy array and driving electronics along with a MR image that shows the array elements, the human skull, and the pig brain.

FIGS. 4A-4B show a treatment envelope indicated by ACE detection in skull 1 (SDR=0.7) and skull 2 (SDR=0.3), respectively. Vertices on the skull mesh are reconstructed from CT scans. On-target cavitation detection is shown, along with pre-focal cavitation detection or no cavitation events.

FIGS. 5A-5D show FIG. 5A) Pre-treatment Gaussian-fitted MR thermometry image overlaid on b=0 dMRI image. FIG. 5B) Post-treatment dMRI image showing the lesion and insert with the MR-thermometry focus and geometric focus. FIG. 5C) dMRI (b=1500) of the square histotripsy lesions in ex vivo bovine treated using different doses (# of pulses below each lesion). FIG. 5D) The AADC value increases with increasing histotripsy doses.

FIGS. 6A-6I show MRI and H&E slices from three pigs. FIGS. 5A-5C: brain H&E shows lesions at targeted locations from pig 1 (left), pig 2 (middle), and pig 3 (right).

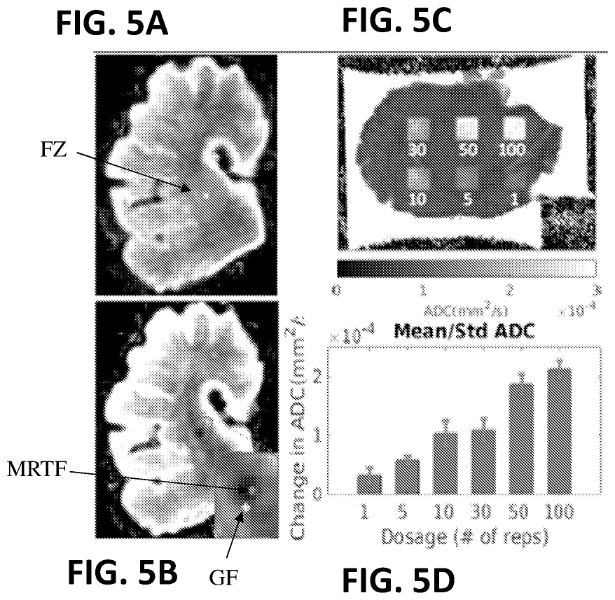

FIGS. 5D-5I: T2- and T2*-weighted MR images from each pig.

FIGS. 7A-7B show Pre- and post-histotripsy dMRI of the 1 cm³ cubic lesion generated in a human cadaveric brain.

FIGS. 8A-8C show example amplitude maps and corresponding incident angle map from skull 2 targeting superior location 10 mm from skull surface, resulting in pre-focal hotspot in the skull. FIG. 8A) Maximum pressure within the focal zone generated by individual elements. FIG. 8B) Pressure contribution from individual elements to the pre-focal hotspot. FIG. 8C) Incident angles on the skull exterior surface from the individual elements.

FIGS. 9A-9D show a demonstration of the new high packing density array construction method. FIG. 9A) CAD model of array element module, showing matching layers, PZT, and housing. FIG. 9B) CAD model of full array showing element arrangement.

FIG. 9C) An assembled transducer module. FIG. 9D) Full 260-element array fabricated using the new rapid prototyping method.

FIGS. 10A-10B are: FIG. 10A) Drawing of the AIR coil and transducer placed around a human head. FIG. 10B) Photograph of the 8-channel AIR coil.

FIGS. 11A-11C show: FIG. 11A) an ACE-based localized cavitation map, acquired transcranially using a transmit-receive capable histotripsy array overlaid on a T2-weighted MR image. FIG. 11B) A cavitation map of representative cases of focal cavitation without off-target cavitation, and FIG. 11C) a skull outer surface cavitation without focal cavitation.

### DETAILED DESCRIPTION

The system, methods and devices of the disclosure may be used for open surgical, minimally invasive surgical (laparoscopic and percutaneous), robotic surgical (integrated into a robotically-enabled medical system), endoscopic or completely transdermal extracorporeal non-invasive acoustic cavitation for the treatment of healthy, diseased and/or injured tissue including but not limited to tissue destruction, cutting, skeletonizing and ablation. Furthermore, due to tissue selective properties, histotripsy may be used to create a cytoskeleton that allows for subsequent tissue regeneration either de novo or through the application of stem cells and other adjuvants. Finally, histotripsy can be used to cause the release of delivered agents such as chemotherapy and immunotherapy by locally causing the release of these agents by the application of acoustic energy to the targets. As will be described below, the acoustic cavitation system may include various sub-systems, including a Cart, Therapy, Integrated Imaging, Robotics, Coupling and Software. The system also may comprise various Other Components, Ancillaries and Accessories, including but not limited to computers, cables and connectors, networking devices, power supplies, displays, drawers/storage, doors, wheels, and various simulation and training tools, etc. All systems, methods and means creating/controlling/delivering histotripsy are considered to be a part of this disclosure, including new related inventions disclosed herein.

Histotripsy therapy is typically guided by ultrasound imaging. Histotripsy can also be guided by MRI. This disclosure describes novel systems (hardware and software) and methods for simultaneous ultrasound and MRI guidance and monitoring for histotripsy treatment. This disclosure describes systems and methods that use magnetic resonance (MR) thermometry combined with low temperature focused ultrasound (FUS) heating or MR-acoustic radiation force imaging (MR-ARFI) to accurately predict a locus of histotripsy cavitation. Further, histotripsy-generated ablation (e.g., brain ablation) can be visualized using diffusion-weighted MRI (dMRI).

This disclosure also provides techniques to enhance MR image quality by integrating ultrasound-translucent RF receive coils into the histotripsy system. Additionally, driving electronics are provided that permit a single amplifier setup to both transmit histotripsy acoustic pulses and receive subsequent acoustic cavitation emission (ACE) signals. This disclosure provides ACE-based quantitative cavitation monitoring to enable real-time treatment ultrasound monitoring at a high frame rate (≥50 Hz) that is practical for histotripsy delivery as well as cavitation mapping of on-target and off-target locations including on a bone (e.g., skull) surface. The cavitation mapping can be complemented by periodic updates from dMRI to provide tissue damage evaluation during histotripsy with both high spatial and temporal resolution.

This disclosure also provides color-encoded cavitation/damage maps based on ACE signals that can be co-registered and superimposed over MR images to form integrated US and MR guidance.

This disclosure also describes a transcranial MR and ultrasound (US) guided Histotripsy (tcMR-USgHt) system. This tcMR-USgHt system can be configured to produce co-registered MRI and US treatment guidance and monitoring at high spatial and temporal resolution and at all intracranial locations.

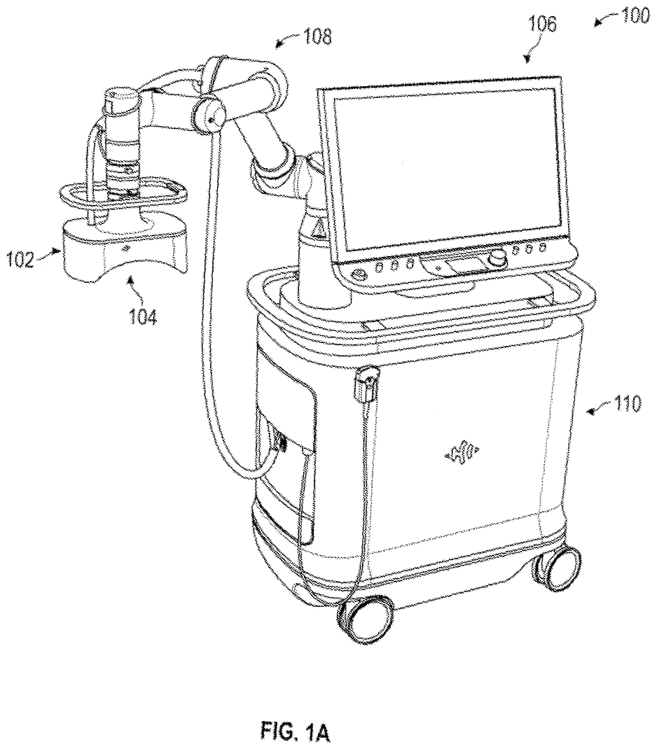

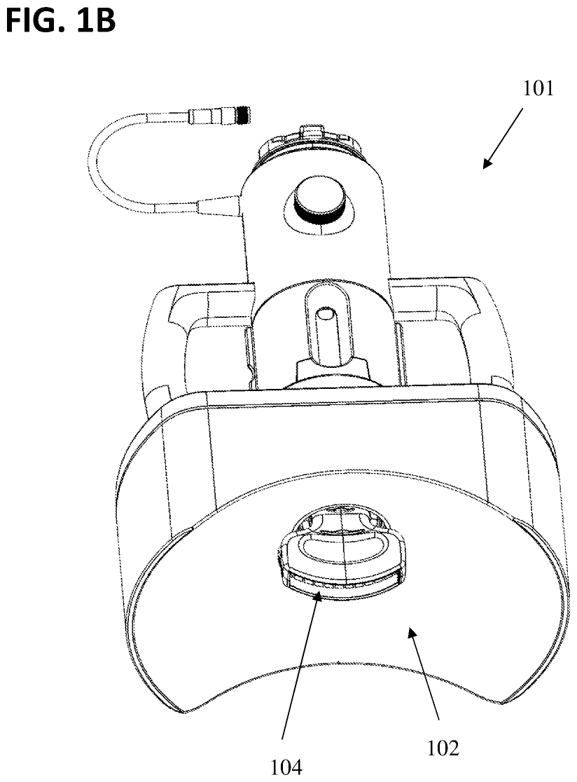

FIG. 1A generally illustrates histotripsy system 100 according to the present disclosure, comprising a therapy transducer 102, an imaging system 104, a display and control panel 106, a robotic positioning arm 108, and a cart 110. The system can further include an ultrasound coupling interface and a source of coupling medium, not shown.

FIG. 1B is a bottom view of a treatment head 101 that includes the therapy transducer 102 and the imaging system 104. As shown, the imaging system can be positioned in the center of the therapy transducer. However, other embodiments can include the imaging system positioned in other locations within the therapy transducer, or even directly integrated into the therapy transducer. In some embodiments, the imaging system is configured to produce real-time imaging at a focal point of the therapy transducer. The system also allows for multiple imaging transducers to be located within the therapy transducer to provide multiple views of the target tissue simultaneously and to integrate these images into a single 3-D image.

The histotripsy system may comprise one or more of various sub-systems, including a Therapy sub-system that can create, apply, focus and deliver acoustic cavitation/histotripsy through one or more therapy transducers, Integrated Imaging sub-system (or connectivity to) allowing real-time visualization of the treatment site and histotripsy effect through-out the procedure, a Robotics positioning sub-system to mechanically and/or electronically steer the therapy transducer, further enabled to connect/support or interact with a Coupling sub-system to allow acoustic coupling between the therapy transducer and the patient, and Software to communicate, control and interface with the system and computer-based control systems (and other external systems) and various Other Components, Ancillaries and Accessories, including one or more user interfaces and displays, and related guided work-flows, all working in part or together. The system may further comprise various fluidics and fluid management components, including but not limited to, pumps, valve and flow controls, temperature and degassing controls, and irrigation and aspiration capabilities, as well as providing and storing fluids. It may also contain various power supplies and protectors.

As described above, the histotripsy system may include integrated imaging. However, in other embodiments, the histotripsy system can be configured to interface with separate imaging systems, such as C-arm, fluoroscope, cone beam CT, MRI, etc., to provide real-time imaging during histotripsy therapy. In some embodiments, the histotripsy system can be sized and configured to fit within a C-arm, fluoroscope, cone beam CT, MRI, etc.

### Cart

The Cart 110 may be generally configured in a variety of ways and form factors based on the specific uses and procedures. In some cases, systems may comprise multiple Carts, configured with similar or different arrangements. In some embodiments, the cart may be configured and arranged to be used in a radiology environment and in some cases in concert with imaging (e.g., CT, cone beam CT and/or MRI scanning). In other embodiments, it may be arranged for use in an operating room and a sterile environment for open surgical or laparoscopic surgical and endoscopic application, or in a robotically enabled operating room, and used alone, or as part of a surgical robotics procedure wherein a surgical robot conducts specific tasks before, during or after use of the system and delivery of acoustic cavitation/histotripsy. As such and depending on the procedure environment based on the aforementioned embodiments, the cart may be positioned to provide sufficient work-space and access to various anatomical locations on the patient (e.g., torso, abdomen, flank, head and neck, etc.), as well as providing work-space for other systems (e.g., anesthesia cart, laparoscopic tower, surgical robot, endoscope tower, etc.).

The Cart may also work with a patient surface (e.g., table or bed) to allow the patient to be presented and repositioned in a plethora of positions, angles and orientations, including allowing changes to such to be made pre, peri and post-procedurally. It may further comprise the ability to interface and communicate with one or more external imaging or image data management and communication systems, not limited to ultrasound, CT, fluoroscopy, cone beam CT, PET, PET/CT, MRI, optical, ultrasound, and image fusion and or image flow, of one or more modalities, to support the procedures and/or environments of use, including physical/mechanical interoperability (e.g., compatible within cone beam CT work-space for collecting imaging data pre, peri and/or post histotripsy) and to provide access to and display of patient medical data including but not limited to laboratory and historical medical record data.

In some embodiments one or more Carts may be configured to work together. As an example, one Cart may comprise a bedside mobile Cart equipped with one or more Robotic arms enabled with a Therapy transducer, and Therapy generator/amplifier, etc., while a companion cart working in concert and at a distance of the patient may comprise Integrated Imaging and a console/display for controlling the Robotic and Therapy facets, analogous to a surgical robot and master/slave configurations.

In some embodiments, the system may comprise a plurality of Carts, all slave to one master Cart, equipped to conduct acoustic cavitation procedures. In some arrangements and cases, one Cart configuration may allow for storage of specific sub-systems at a distance reducing operating room clutter, while another in concert Cart may comprise essentially bedside sub-systems and componentry (e.g., delivery system and therapy).

One can envision a plethora of permutations and configurations of Cart design, and these examples are in no way limiting the scope of the disclosure.

### Histotripsy

Histotripsy comprises short, high amplitude, focused ultrasound pulses to generate a dense, energetic, “bubble cloud”, capable of the targeted fractionation and destruction of tissue. Histotripsy is capable of creating controlled tissue erosion when directed at a tissue interface, including tissue/fluid interfaces, as well as well-demarcated tissue fractionation and destruction, at sub-cellular levels, when it is targeted at bulk tissue. Unlike other forms of ablation, including thermal and radiation-based modalities, histotripsy does not rely on heat cold or ionizing (high) energy to treat tissue. Instead, histotripsy uses acoustic cavitation generated at the focus to mechanically effect tissue structure, and in some cases liquefy, suspend, solubilize and/or destruct tissue into sub-cellular components.

Histotripsy can be applied in various forms, including: 1) Intrinsic-Threshold Histotripsy: Delivers pulses with a 1-2 cycles of high amplitude negative/tensile phase pressure exceeding the intrinsic threshold to generate cavitation in the medium (e.g., ~24-28 MPa for water-based soft tissue), 2) Shock-Scattering Histotripsy: Delivers typically pulses 3-20 cycles in duration. The shockwave (positive/compressive phase) scattered from an initial individual microbubble generated forms inverted shockwave, which constructively interfere with the incoming negative/tensile phase to form high amplitude negative/rarefactional phase exceeding the intrinsic threshold. In this way, a cluster of cavitation microbubbles is generated. The amplitude of the tensile phases of the pulses is sufficient to cause bubble nuclei in the medium to undergo inertial cavitation within the focal zone throughout the duration of the pulse. These nuclei scatter the incident shockwaves, which invert and constructively interfere with the incident wave to exceed the threshold for intrinsic nucleation, and 3) Boiling Histotripsy: Employs pulses roughly 1-20 ms in duration.

Absorption of the shocked pulse rapidly heats the medium, thereby reducing the threshold for intrinsic nuclei. Once this intrinsic threshold coincides with the peak negative pressure of the incident wave, boiling bubbles form at the focus.

The large pressure generated at the focus causes a cloud of acoustic cavitation bubbles to form above certain thresholds, which creates localized stress and strain in the tissue and mechanical breakdown without significant heat deposition. At pressure levels where cavitation is not generated, minimal effect is observed on the tissue at the focus. This cavitation effect is observed only at pressure levels significantly greater than those which define the inertial cavitation threshold in water for similar pulse durations, on the order of 10 to 30 MPa peak negative pressure.

Histotripsy may be performed in multiple ways and under different parameters. It may be performed totally non-invasively by acoustically coupling a focused ultrasound transducer over the skin of a patient and transmitting acoustic pulses transcutaneously through overlying (and intervening) tissue to the focal zone (treatment zone and site). The application of histotripsy is not limited to a transdermal approach but can be applied through any means that allows contact of the transducer with tissue including open surgical laparoscopic surgical, percutaneous and robotically mediated surgical procedures. It may be further targeted, planned, directed and observed under direct visualization, via ultrasound imaging, given the bubble clouds generated by histotripsy may be visible as highly dynamic, echogenic regions on, for example, B Mode ultrasound images, allowing continuous visualization through its use (and related procedures). Likewise, the treated and fractionated tissue shows a dynamic change in echogenicity (typically a reduction), which can be used to evaluate, plan, observe and monitor treatment.

Generally, in histotripsy treatments, ultrasound pulses with 1 or more acoustic cycles are applied, and the bubble cloud formation relies on the pressure release scattering of the positive shock fronts (sometimes exceeding 100 MPa, P+) from initially initiated, sparsely distributed bubbles (or a single bubble). This is referred to as the “shock scattering mechanism”.

This mechanism depends on one (or a few sparsely distributed) bubble(s) initiated with the initial negative half cycle(s) of the pulse at the focus of the transducer. A cloud of microbubbles then forms due to the pressure release backscattering of the high peak positive shock fronts from these sparsely initiated bubbles. These back-scattered high-amplitude rarefactional waves exceed the intrinsic threshold thus producing a localized dense bubble cloud. Each of the following acoustic cycles then induces further cavitation by the backscattering from the bubble cloud surface, which grows towards the transducer. As a result, an elongated dense bubble cloud growing along the acoustic axis opposite the ultrasound propagation direction is observed with the shock scattering mechanism. This shock scattering process makes the bubble cloud generation not only dependent on the peak negative pressure, but also the number of acoustic cycles and the amplitudes of the positive shocks. Without at least one intense shock front developed by nonlinear propagation, no dense bubble clouds are generated when the peak negative half-cycles are below the intrinsic threshold.

When ultrasound pulses less than 2 cycles are applied, shock scattering can be minimized, and the generation of a dense bubble cloud depends on the negative half cycle(s) of the applied ultrasound pulses exceeding an “intrinsic threshold” of the medium. This is referred to as the “intrinsic threshold mechanism”.

This threshold can be in the range of 26-30 MPa for soft tissues with high water content, such as tissues in the human body. In some embodiments, using this intrinsic threshold mechanism, the spatial extent of the lesion may be well-defined and more predictable. With peak negative pressures (P−) not significantly higher than this threshold, sub-wavelength reproducible lesions as small as half of the −6 dB beam width of a transducer may be generated.

With high-frequency Histotripsy pulses, the size of the smallest reproducible lesion becomes smaller, which is beneficial in applications that require precise lesion generation. However, high-frequency pulses are more susceptible to attenuation and aberration, rendering problematical treatments at a larger penetration depth (e.g., ablation deep in the body) or through a highly aberrative medium (e.g., transcranial procedures, or procedures in which the pulses are transmitted through bone(s)). Histotripsy may further also be applied as a low-frequency “pump” pulse (typically <2 cycles and having a frequency between 100 kHz and 1 MHz) can be applied together with a high-frequency “probe” pulse (typically <2 cycles and having a frequency greater than 2 MHz, or ranging between 2 MHz and 10 MHz) wherein the peak negative pressures of the low and high-frequency pulses constructively interfere to exceed the intrinsic threshold in the target tissue or medium. The low-frequency pulse, which is more resistant to attenuation and aberration, can raise the peak negative pressure P-level for a region of interest (ROI), while the high-frequency pulse, which provides more precision, can pin-point a targeted location within the ROI and raise the peak negative pressure P-above the intrinsic threshold. This approach may be referred to as “dual frequency”, “dual beam histotripsy” or “parametric histotripsy.”

Additional systems, methods and parameters to deliver optimized histotripsy, using shock scattering, intrinsic threshold, and various parameters enabling frequency compounding and bubble manipulation, are herein included as part of the system and methods disclosed herein, including additional means of controlling said histotripsy effect as pertains to steering and positioning the focus, and concurrently managing tissue effects (e.g., prefocal thermal collateral damage) at the treatment site or within intervening tissue. Further, it is disclosed that the various systems and methods, which may include a plurality of parameters, such as but not limited to, frequency, operating frequency, center frequency, pulse repetition frequency, pulses, bursts, number of pulses, cycles, length of pulses, amplitude of pulses, pulse period, delays, burst repetition frequency, sets of the former, loops of multiple sets, loops of multiple and/or different sets, sets of loops, and various combinations or permutations of, etc., are included as a part of this disclosure, including future envisioned embodiments of such.

### Therapy Components

The Therapy sub-system may work with other sub-systems to create, optimize, deliver, visualize, monitor and control acoustic cavitation, also referred to herein and in following as “histotripsy”, and its derivatives of, including boiling histotripsy and other thermal high frequency ultrasound approaches. It is noted that the disclosed inventions may also further benefit other acoustic therapies that do not comprise a cavitation, mechanical or histotripsy component. The therapy sub-system can include, among other features, an ultrasound therapy transducer and a pulse generator system configured to deliver ultrasound pulses into tissue. The pulse generator can be incorporated into, for example, the therapy cart such as within cart 110 of FIG. 1A.

In order to create and deliver histotripsy and derivatives of histotripsy, the therapy sub-system may also comprise components, including but not limited to, one or more function generators, amplifiers, therapy transducers and power supplies.

The therapy transducer can comprise a single element or multiple elements configured to be excited with high amplitude electric pulses (>1000V or any other voltage that can cause harm to living organisms). The amplitude necessary to drive the therapy transducers for Histotripsy vary depending on the design of the transducer and the materials used (e.g., solid or polymer/piezoelectric composite including ceramic or single crystal) and the transducer center frequency which is directly proportional to the thickness of the piezo-electric material. Transducers therefore operating at a high frequency require lower voltage to produce a given surface pressure than is required by low frequency therapy transducers. In some embodiments, the transducer elements are formed using a piezoelectric-polymer composite material or a solid piezoelectric material. Further, the piezoelectric material can be of polycrystalline/ceramic or single crystalline formulation. In some embodiments the transducer elements can be formed using silicon using MEMs technology, including CMUT and PMUT designs.

In some embodiments, the function generator may comprise a field programmable gate array (FPGA) or other suitable function generator. The FPGA may be configured with parameters disclosed previously herein, including but not limited to frequency, pulse repetition frequency, bursts, burst numbers, where bursts may comprise pulses, numbers of pulses, length of pulses, pulse period, delays, burst repetition frequency or period, where sets of bursts may comprise a parameter set, where loop sets may comprise various parameter sets, with or without delays, or varied delays, where multiple loop sets may be repeated and/or new loop sets introduced, of varied time delay and independently controlled, and of various combinations and permutations of such, overall and throughout.

In some embodiments, the generator or amplifier may be configured to be a universal single-cycle or multi-cycle pulse generator, and to support driving via Class D or inductive driving, as well as across all envisioned clinical applications, use environments, also discussed in part later in this disclosure. In other embodiments, the class D or inductive current driver may be configured to comprise transformer and/or auto-transformer driving circuits to further provide step up/down components, and in some cases, to preferably allow a step up in the amplitude. They may also comprise specific protective features, to further support the system, and provide capability to protect other parts of the system (e.g., therapy transducer and/or amplifier circuit components) and/or the user, from various hazards, including but not limited to, electrical safety hazards, which may potentially lead to use environment, system and therapy system, and user harms, damage or issues.

Disclosed generators may allow and support the ability of the system to select, vary and control various parameters (through enabled software tools), including, but not limited to those previously disclosed, as well as the ability to start/stop therapy, set and read voltage level, pulse and/or burst repetition frequency, number of cycles, duty ratio, channel enabled and delay, etc., modulate pulse amplitude on a fast time-scale independent of a high voltage supply, and/or other service, diagnostic or treatment features.

In some embodiments, the Therapy sub-system and/or components of, such as the amplifier, may comprise further integrated computer processing capability and may be networked, connected, accessed, and/or be removable/portable, modular, and/or exchangeable between systems, and/or driven/commanded from/by other systems, or in various combinations. Other systems may include other acoustic cavitation/histotripsy, HIFU, HITU, radiation therapy, radiofrequency, microwave, and cryoablation systems, navigation and localization systems, open surgical, laparoscopic, single incision/single port, endoscopic and non-invasive surgical robots, laparoscopic or surgical towers comprising other energy-based or vision systems, surgical system racks or booms, imaging carts, etc.

In some embodiments, one or more amplifiers may comprise a Class D amplifier and related drive circuitry including matching network components. Depending on the transducer element electric impedance and choice of the matching network components (e.g., an LC circuit made of an inductor L1 in series and the capacitor C1 in parallel), the combined impedance can be aggressively set low in order to have high amplitude electric waveform necessary to drive the transducer element. The maximum amplitude that Class D amplifiers is dependent on the circuit components used, including the driving MOSFET/IGBT transistors, matching network components or inductor, and transformer or autotransformer, and of which may be typically in the low kV (e.g., 1-3 kV) range.

Therapy transducer element(s) are excited with an electrical waveform with an amplitude (voltage) to produce a pressure output sufficient for Histotripsy therapy. The excitation electric field can be defined as the necessary waveform voltage per thickness of the piezoelectric element. For example, because a piezoelectric element operating at 1 MHz transducer is half the thickness of an equivalent 500 kHz element, it will require half the voltage to achieve the same electric field and surface pressure.

The Therapy sub-system may also comprise therapy transducers of various designs and working parameters, supporting use in various procedures (and procedure settings). Systems may be configured with one or more therapy transducers, that may be further interchangeable, and work with various aspects of the system in similar or different ways (e.g., may interface to a robotic arm using a common interface and exchange feature, or conversely, may adapt to work differently with application specific imaging probes, where different imaging probes may interface and integrate with a therapy transducer in specifically different ways).

Therapy transducers may be configured of various parameters that may include size, shape (e.g., rectangular or round; anatomically curved housings, etc.), geometry, focal length, number of elements, size of elements, distribution of elements (e.g., number of rings, size of rings for annular patterned transducers), frequency, enabling electronic beam steering, etc. Transducers may be composed of various materials (e.g., piezoelectric, silicon, etc.), form factors and types (e.g., machined elements, chip-based, etc.) and/or by various methods of fabrication of.

Transducers may be designed and optimized for clinical applications (e.g., abdominal tumors, peripheral vascular disease, fat ablation, etc.) and desired outcomes (e.g., acoustic cavitation/histotripsy without thermal injury to intervening tissue), and affording a breadth of working ranges, including relatively shallow and superficial targets (e.g., thyroid or breast nodules), versus, deeper or harder to reach targets, such as central liver or brain tumors. They may be configured to enable acoustic cavitation/histotripsy under various parameters and sets of, as enabled by the aforementioned system components (e.g., function generator and amplifier, etc.), including but not limited to frequency, pulse repetition rate, pulses, number of pulses, pulse length, pulse period, delays, repetitions, sync delays, sync period, sync pulses, sync pulse delays, various loop sets, others, and permutations of. The transducer may also be designed to allow for the activation of a drug payload either deposited in tissue through various means including injection, placement or delivery in micelle or nanostructures.

### Integrated Imaging

The disclosed system may comprise various imaging modalities to allow users to visualize, monitor and collect/use feedback of the patient's anatomy, related regions of interest and treatment/procedure sites, as well as surrounding and intervening tissues to assess, plan and conduct procedures, and adjust treatment parameters as needed. Imaging modalities may comprise various ultrasound, x-ray, CT, MRI, PET, fluoroscopy, optical, contrast or agent enhanced versions, and/or various combinations of. It is further disclosed that various image processing and characterization technologies may also be utilized to afford enhanced visualization and user decision making. These may be selected or commanded manually by the user or in an automated fashion by the system. The system may be configured to allow side by side, toggling, overlays, 3D reconstruction, segmentation, registration, multi-modal image fusion, image flow, and/or any methodology affording the user to identify, define and inform various aspects of using imaging during the procedure, as displayed in the various system user interfaces and displays. Examples may include locating, displaying and characterizing regions of interest, organ systems, potential treatment sites within, with on and/or surrounding organs or tissues, identifying critical structures such as ducts, vessels, nerves, ureters, fissures, capsules, tumors, tissue trauma/injury/disease, other organs, connective tissues, etc., and/or in context to one another, of one or more (e.g., tumor draining lymphatics or vasculature; or tumor proximity to organ capsule or underlying other organ), as unlimited examples.

Systems may be configured to include onboard integrated imaging hardware, software, sensors, probes and wetware, and/or may be configured to communicate and interface with external imaging and image processing systems. The aforementioned components may be also integrated into the system's Therapy sub-system components wherein probes, imaging arrays, or the like, and electrically, mechanically or electromechanically integrated into therapy transducers. This may afford, in part, the ability to have geometrically aligned imaging and therapy, with the therapy directly within the field of view, and in some cases in line, with imaging. In some embodiments, this integration may comprise a fixed orientation of the imaging capability (e.g., imaging probe) in context to the therapy transducer. In other embodiments, the imaging solution may be able to move or adjust its position, including modifying angle, extension (e.g., distance from therapy transducer or patient), rotation (e.g., imaging plane in example of an ultrasound probe) and/or other parameters, including moving/adjusting dynamically while actively imaging. The imaging component or probe may be encoded so its orientation and position relative to another aspect of the system, such as the therapy transducer, and/or robotically-enabled positioning component may be determined.

In one embodiment, the system may comprise onboard ultrasound, further configured to allow users to visualize, monitor and receive feedback for procedure sites through the system displays and software, including allowing ultrasound imaging and characterization (and various forms of), ultrasound guided planning and ultrasound guided treatment, all in real-time. The system may be configured to allow users to manually, semi-automated or in fully automated means image the patient (e.g., by hand or using a robotically-enabled imager).

In some embodiments, imaging feedback and monitoring can include monitoring changes in: backscatter from bubble clouds; speckle reduction in backscatter; backscatter speckle statistics; mechanical properties of tissue (i.e., elastography); tissue perfusion (i.e., ultrasound contrast); shear wave propagation; acoustic emissions, electrical impedance tomography, and/or various combinations of, including as displayed or integrated with other forms of imaging (e.g., CT or MRI).

In some embodiments, imaging including feedback and monitoring from backscatter from bubble clouds, may be used as a method to determine immediately if the histotripsy process has been initiated, is being properly maintained, or even if it has been extinguished. For example, this method enables continuously monitored in real time drug delivery, tissue erosion, and the like. The method also can provide feedback permitting the histotripsy process to be initiated at a higher intensity and maintained at a much lower intensity. For example, backscatter feedback can be monitored by any transducer or ultrasonic imager. By measuring feedback for the therapy transducer, an accessory transducer can send out interrogation pulses or be configured to passively detect cavitation. Moreover, the nature of the feedback received can be used to adjust acoustic parameters (and associated system parameters) to optimize the drug delivery and/or tissue erosion process.

In some embodiments, imaging including feedback and monitoring from backscatter, and speckle reduction, may be configured in the system.

For systems comprising feedback and monitoring via backscattering, and as means of background, as tissue is progressively mechanically subdivided, in other words homogenized, disrupted, or eroded tissue, this process results in changes in the size and distribution of acoustic scatter. At some point in the process, the scattering particle size and density is reduced to levels where little ultrasound is scattered, or the amount scattered is reduced significantly. This results in a significant reduction in speckle, which is the coherent constructive and destructive interference patterns of light and dark spots seen on images when coherent sources of illumination are used; in this case, ultrasound. After some treatment time, the speckle reduction results in a dark area in the therapy volume. Since the amount of speckle reduction is related to the amount of tissue subdivision, it can be related to the size of the remaining tissue fragments. When this size is reduced to sub-cellular levels, no cells are assumed to have survived. So, treatment can proceed until a desired speckle reduction level has been reached. Speckle is easily seen and evaluated on standard ultrasound imaging systems. Specialized transducers and systems, including those disclosed herein, may also be used to evaluate the backscatter changes.

Further, systems comprising feedback and monitoring via speckle, and as means of background, an image may persist from frame to frame and change very little as long as the scatter distribution does not change and there is no movement of the imaged object. However, long before the scatters are reduced enough in size to cause speckle reduction, they may be changed sufficiently to be detected by signal processing and other means. This family of techniques can operate as detectors of speckle statistics changes. For example, the size and position of one or more speckles in an image will begin to decorrelate before observable speckle reduction occurs. Speckle decorrelation, after appropriate motion compensation, can be a sensitive measure of the mechanical disruption of the tissues, and thus a measure of therapeutic efficacy. This feedback and monitoring technique may permit early observation of changes resulting from the acoustic cavitation/histotripsy process and can identify changes in tissue before substantial or complete tissue effect (e.g., erosion occurs). In one embodiment, this method may be used to monitor the acoustic cavitation/histotripsy process for enhanced drug delivery where treatment sites/tissue is temporally disrupted, and tissue damage/erosion is not desired. In other embodiments, this may comprise speckle decorrelation by movement of scatters in an increasingly fluidized therapy volume. For example, in the case where partial or complete tissue erosion is desired.

For systems comprising feedback and monitoring via elastography, and as means of background, as treatment sites/tissue are further subdivided per an acoustic cavitation/histotripsy effect (homogenized, disrupted, or eroded), its mechanical properties change from a soft but interconnected solid to a viscous fluid or paste with few long-range interactions. These changes in mechanical properties can be measured by various imaging modalities including MRI and ultrasound imaging systems. For example, an ultrasound pulse can be used to produce a force (i.e., a radiation force) on a localized volume of tissue. The tissue response (displacements, strains, and velocities) can change significantly during histotripsy treatment allowing the state of tissue disruption to be determined by imaging or other quantitative means.

Systems may also comprise feedback and monitoring via shear wave propagation changes. As means of background, the subdivision of tissues makes the tissue more fluid and less solid and fluid systems generally do not propagate shear waves. Thus, the extent of tissue fluidization provides opportunities for feedback and monitoring of the histotripsy process. For example, ultrasound and MRI imaging systems can be used to observe the propagation of shear waves. The extinction of such waves in a treated volume is used as a measure of tissue destruction or disruption. In one system embodiment, the system and supporting sub-systems may be used to generate and measure the interacting shear waves. For example, two adjacent ultrasound foci might perturb tissue by pushing it in certain ways. If adjacent foci are in a fluid, no shear waves propagate to interact with each other. If the tissue is not fluidized, the interaction would be detected with external means, for example, by a difference frequency only detected when two shear waves interact nonlinearly, with their disappearance correlated to tissue damage. As such, the system may be configured to use this modality to enhance feedback and monitoring of the acoustic cavitation/histotripsy procedure.

For systems comprising feedback and monitoring via acoustic emission, and as means of background, as a tissue volume is subdivided, its effect on acoustic cavitation/histotripsy (e.g., the bubble cloud here) is changed. For example, bubbles may grow larger and have a different lifetime and collapse changing characteristics in intact versus fluidized tissue. Bubbles may also move and interact after tissue is subdivided producing larger bubbles or cooperative interaction among bubbles, all of which can result in changes in acoustic emission. These emissions can be heard during treatment and they change during treatment. Analysis of these changes, and their correlation to therapeutic efficacy, enables monitoring of the progress of therapy, and may be configured as a feature of the system.

For systems comprising feedback and monitoring via electrical impedance tomography, and as means of background, an impedance map of a therapy site can be produced based upon the spatial electrical characteristics throughout the therapy site. Imaging of the conductivity or permittivity of the therapy site of a patient can be inferred from taking skin surface electrical measurements. Conducting electrodes are attached to a patient's skin and small alternating currents are applied to some or all of the electrodes. One or more known currents are injected into the surface and the voltage is measured at a number of points using the electrodes. The process can be repeated for different configurations of applied current. The resolution of the resultant image can be adjusted by changing the number of electrodes employed. A measure of the electrical properties of the therapy site within the skin surface can be obtained from the impedance map, and changes in and location of the acoustic cavitation/histotripsy (e.g., bubble cloud, specifically) and histotripsy process can be monitored using this as configured in the system and supporting sub-systems.

The user may be allowed to further select, annotate, mark, highlight, and/or contour, various regions of interest or treatment sites, and defined treatment targets (on the image(s)), of which may be used to command and direct the system where to image, test and/or treat, through the system software and user interfaces and displays. In some arrangements, the user may use a manual ultrasound probe (e.g., diagnostic hand-held probe) to conduct the procedure. In another arrangement, the system may use a robot and/or electromechanical positioning system to conduct the procedure, as directed and/or automated by the system, or conversely, the system can enable combinations of manual and automated uses.

The system may further include the ability to conduct image registration, including imaging and image data set registration to allow navigation and localization of the system to the patient, including the treatment site (e.g., tumor, critical structure, bony anatomy, anatomy and identifying features of, etc.). In one embodiment, the system allows the user to image and identify a region of interest, for example the liver, using integrated ultrasound, and to select and mark a tumor (or surrogate marker of) comprised within the liver through/displayed in the system software, and wherein said system registers the image data to a coordinate system defined by the system, that further allows the system's Therapy and Robotics sub-systems to deliver synchronized acoustic cavitation/histotripsy to said marked tumor. The system may comprise the ability to register various image sets, including those previously disclosed, to one another, as well as to afford navigation and localization (e.g., of a therapy transducer to a CT or MRI/ultrasound fusion image with the therapy transducer and Robotics sub-system tracking to said image).

The system may also comprise the ability to work in a variety of interventional, endoscopic and surgical environments, including alone and with other systems (surgical/laparoscopic towers, vision systems, endoscope systems and towers, ultrasound enabled endoscopic ultrasound (flexible and rigid), percutaneous/endoscopic/laparoscopic and minimally invasive navigation systems (e.g., optical, electromagnetic, shape-sensing, ultrasound-enabled, etc.), of also which may work with, or comprise various optical imaging capabilities (e.g., fiber and or digital). The disclosed system may be configured to work with these systems, in some embodiments working alongside them in concert, or in other embodiments where all or some of the system may be integrated into the above systems/platforms (e.g., acoustic cavitation/histotripsy-enabled endoscope system or laparoscopic surgical robot). In many of these environments, a therapy transducer may be utilized at or around the time of use, for example, of an optically guided endoscope/bronchoscope, or as another example, at the time a laparoscopic robot (e.g., Intuitive Da Vinci*Xi system) is viewing/manipulating a tissue/treatment site. Further, these embodiments and examples may include where said other systems/platforms are used to deliver (locally) fluid to enable the creation of a man-made acoustic window, where on under normal circumstances may not exist (e.g., fluidizing a segment or lobe of the lung in preparation for acoustic cavitation/histotripsy via non-invasive transthoracic treatment (e.g., transducer externally placed on/around patient). Systems disclosed herein may also comprise all or some of their sub-system hardware packaged within the other system cart/console/systems described here (e.g., acoustic cavitation/histotripsy system and/or sub-systems integrated and operated from said navigation or laparoscopic system).

The system may also be configured, through various aforementioned parameters and other parameters, to display real-time visualization of a bubble cloud in a spatial-temporal manner, including the resulting tissue effect peri/post-treatment from tissue/bubble cloud interaction, wherein the system can dynamically image and visualize, and display, the bubble cloud, and any changes to it (e.g., decreasing or increasing echogenicity), which may include intensity, shape, size, location, morphology, persistence, etc. These features may allow users to continuously track and follow the treatment in real-time in one integrated procedure and interface/system, and confirm treatment safety and efficacy on the fly (versus other interventional or surgical modalities, which either require multiple procedures to achieve the same, or where the treatment effect is not visible in real-time (e.g., radiation therapy), or where it is not possible to achieve such (e.g., real-time visualization of local tissue during thermal ablation), and/or where the other procedure further require invasive approaches (e.g., incisions or punctures) and iterative imaging in a scanner between procedure steps (e.g., CT or MRI scanning). The above disclosed systems, sub-systems, components, modalities, features and work-flows/methods of use may be implemented in an unlimited fashion through enabling hardware, software, user interfaces and use environments, and future improvements, enhancements and inventions in this area are considered as included in the scope of this disclosure, as well as any of the resulting data and means of using said data for analytics, artificial intelligence or digital health applications and systems.

### Robotics

They system may comprise various Robotic sub-systems and components, including but not limited to, one or more robotic arms and controllers, which may further work with other sub-systems or components of the system to deliver and monitor acoustic cavitation/histotripsy. As previously discussed herein, robotic arms and control systems may be integrated into one or more Cart configurations.

For example, one system embodiment may comprise a Cart with an integrated robotic arm and control system, and Therapy, Integrated Imaging and Software, where the robotic arm and other listed sub-systems are controlled by the user through the form factor of a single bedside Cart.

In other embodiments, the Robotic sub-system may be configured in one or more separate Carts, that may be a driven in a master/slave configuration from a separate master or Cart, wherein the robotically-enabled Cart is positioned bed/patient-side, and the Master is at a distance from said Cart.

Disclosed robotic arms may be comprised of a plurality of joints, segments, and degrees of freedom and may also include various integrated sensor types and encoders, implemented for various use and safety features. Sensing technologies and data may comprise, as an example, vision, potentiometers, position/localization, kinematics, force, torque, speed, acceleration, dynamic loading, and/or others. In some cases, sensors may be used for users to direct robot commands (e.g., hand gesture the robot into a preferred set up position, or to dock home). Additional details on robotic arms can be found in U.S. Patent Pub. No. 2013/0255426 A1 to Kassow et al. which is disclosed herein by reference in its entirety.

The robotic arm receives control signals and commands from the robotic control system, which may be housed in a Cart. The system may be configured to provide various functionalities, including but not limited to, position, tracking, patterns, triggering, and events/actions.

Position may be configured to comprise fixed positions, pallet positions, time-controlled positions, distance-controlled positions, variable-time controlled positions, variable-distance controlled positions.

Tracking may be configured to comprise time-controlled tracking and/or distance-controlled tracking.

The patterns of movement may be configured to comprise intermediate positions or waypoints, as well as sequence of positions, through a defined path in space.

Triggers may be configured to comprise distance measuring means, time, and/or various sensor means including those disclosed herein, and not limited to, visual/imaging-based, force, torque, localization, energy/power feedback and/or others.

Events/actions may be configured to comprise various examples, including proximity-based (approaching/departing a target object), activation or de-activation of various end-effectors (e.g., therapy transducers), starting/stopping/pausing sequences of said events, triggering or switching between triggers of events/actions, initiating patterns of movement and changing/toggling between patterns of movement, and/or time-based and temporal over the defined work and time-space.

In one embodiment, the system comprises a three degree of freedom robotic positioning system, enabled to allow the user (through the software of the system and related user interfaces), to micro-position a therapy transducer through X, Y, and Z coordinate system, and where gross macro-positioning of the transducer (e.g., aligning the transducer on the patient's body) is completed manually. In some embodiments, the robot may comprise 6 degrees of freedom including X, Y, Z, and pitch, roll and yaw. In other embodiments, the Robotic sub-system may comprise further degrees of freedom, that allow the robot arm supporting base to be positioned along a linear axis running parallel to the general direction of the patient surface, and/or the supporting base height to be adjusted up or down, allowing the position of the robotic arm to be modified relative to the patient, patient surface, Cart, Coupling sub-system, additional robots/robotic arms and/or additional surgical systems, including but not limited to, surgical towers, imaging systems, endoscopic/laparoscopic systems, and/or other.

One or more robotic arms may also comprise various features to assist in maneuvering and modifying the arm position, manually or semi-manually, and of which said features may interface on or between the therapy transducer and the most distal joint of the robotic arm. In some embodiments, the feature is configured to comprise a handle allowing maneuvering and manual control with one or more hands. The handle may also be configured to include user input and electronic control features of the robotic arm, to command various drive capabilities or modes, to actuate the robot to assist in gross or fine positioning of the arm (e.g., activating or deactivating free drive mode). The work-flow for the initial positioning of the robotic arm and therapy head can be configured to allow either first positioning the therapy transducer/head in the coupling solution, with the therapy transducer directly interfaced to the arm, or in a different work-flow, allowing the user to set up the coupling solution first, and enabling the robot arm to be interfaced to the therapy transducer/coupling solution as a later/terminal set up step.

In some embodiments, the one or more robotic arms or other features of the robotic sub-systems may include sensors or other features configured to measure, determine, or predict the force(s) acting against the robotic arm(s) and/or the therapy transducer array coupled to the robotic arm(s). These sensors can include force sensors or force transducers not limited to load cells, pneumatic load cells, capacitive load cells, strain gauge load cells, hydraulic load cells, etc. In some implementations, the force sensors can be disposed on or in the robotic arm(s), on or in the transducer array or therapy probe, on or in the coupling linkages between the transducer array and robotic arm, or in any other location within the system, including the robotics sub-system, where a force sensor or sensors would be adapted and configured to measure the force applied against the robotic arm or the transducer array. Additionally, these force sensors can be electronically or operatively coupled to any of the control systems described herein, including electronic controllers, robotic positioning systems, navigation systems, or any other cpus, processors, or controllers configured to control the operation of the transducer array, robotics sub-system, or any other sub-system during therapy.

In some embodiments, the robotic arm may comprise a robotic arm on a laparoscopic, single port, endoscopic, hybrid or combination of, and/or other robot, wherein said robot of the system may be a slave to a master that controls said arm, as well as potentially a plurality of other arms, equipped to concurrently execute other tasks (vision, imaging, grasping, cutting, ligating, sealing, closing, stapling, ablating, suturing, marking, etc.), including actuating one or more laparoscopic arms (and instruments) and various histotripsy system components. For example, a laparoscopic robot may be utilized to prepare the surgical site, including manipulating organ position to provide more ideal acoustic access and further stabilizing said organ in some cases to minimize respiratory motion. In conjunction and parallel to this, a second robotic arm may be used to deliver non-invasive acoustic cavitation through a body cavity, as observed under real-time imaging from the therapy transducer (e.g., ultrasound) and with concurrent visualization via a laparoscopic camera. In other related aspects, a similar approach may be utilized with a combination of an endoscopic and non-invasive approach, and further, with a combination of an endoscopic, laparoscopic and non-invasive approach.

### Software

The system may comprise various software applications, features and components which allow the user to interact, control and use the system for a plethora of clinical applications. The Software may communicate and work with one or more of the sub-systems, including but not limited to Therapy, Integrated Imaging, Robotics and Other Components, Ancillaries and Accessories of the system.

Overall, in no specific order of importance, the software may provide features and support to initialize and set up the system, service the system, communicate and import/export/store data, modify/manipulate/configure/control/command various settings and parameters by the user, mitigate safety and use-related risks, plan procedures, provide support to various configurations of transducers, robotic arms and drive systems, function generators and amplifier circuits/slaves, test and treatment ultrasound sequences, transducer steering and positioning (electromechanical and electronic beam steering, etc.), treatment patterns, support for imaging and imaging probes, manual and electromechanical/robotically-enabling movement of, imaging support for measuring/characterizing various dimensions within or around procedure and treatment sites (e.g., depth from one anatomical location to another, etc., pre-treatment assessments and protocols for measuring/characterizing in situ treatment site properties and conditions (e.g., acoustic cavitation/histotripsy thresholds and heterogeneity of), targeting and target alignment, calibration, marking/annotating, localizing/navigating, registering, guiding, providing and guiding through work-flows, procedure steps, executing treatment plans and protocols autonomously, autonomously and while under direct observation and viewing with real-time imaging as displayed through the software, including various views and viewports for viewing, communication tools (video, audio, sharing, etc.), troubleshooting, providing directions, warnings, alerts, and/or allowing communication through various networking devices and protocols. It is further envisioned that the software user interfaces and supporting displays may comprise various buttons, commands, icons, graphics, text, etc., that allow the user to interact with the system in a user-friendly and effective manner, and these may be presented in an unlimited number of permutations, layouts and designs, and displayed in similar or different manners or feature sets for systems that may comprise more than one display (e.g., touch screen monitor and touch pad), and/or may network to one or more external displays or systems (e.g., another robot, navigation system, system tower, console, monitor, touch display, mobile device, tablet, etc.).

The software, as a part of a representative system, including one or more computer processors, may support the various aforementioned function generators (e.g., FPGA), amplifiers, power supplies and therapy transducers. The software may be configured to allow users to select, determine and monitor various parameters and settings for acoustic cavitation/histotripsy, and upon observing/receiving feedback on performance and conditions, may allow the user to stop/start/modify said parameters and settings.

The software may be configured to allow users to select from a list or menu of multiple transducers and support the auto-detection of said transducers upon connection to the system (and verification of the appropriate sequence and parameter settings based on selected application). In other embodiments, the software may update the targeting and amplifier settings (e.g., channels) based on the specific transducer selection. The software may also provide transducer recommendations based on pre-treatment and planning inputs. Conversely, the software may provide error messages or warnings to the user if said therapy transducer, amplifier and/or function generator selections or parameters are erroneous, yield a fault or failure. This may further comprise reporting the details and location of such.

In addition to above, the software may be configured to allow users to select treatment sequences and protocols from a list or menu, and to store selected and/or previous selected sequences and protocols as associated with specific clinical uses or patient profiles. Related profiles may comprise any associated patient, procedure, clinical and/or engineering data, and maybe used to inform, modify and/or guide current or future treatments or procedures/interventions, whether as decision support or an active part of a procedure itself (e.g., using serial data sets to build and guide new treatments).

As a part of planning or during the treatment, the software (and in working with other components of the system) may allow the user to evaluate and test acoustic cavitation/histotripsy thresholds at various locations in a user-selected region of interest or defined treatment area/volume, to determine the minimum cavitation thresholds throughout said region or area/volume, to ensure treatment parameters are optimized to achieve, maintain and dynamically control acoustic cavitation/histotripsy. In one embodiment, the system allows a user to manually evaluate and test threshold parameters at various points. Said points may include those at defined boundary, interior to the boundary and center locations/positions, of the selected region of interest and treatment area/volume, and where resulting threshold measurements may be reported/displayed to the user, as well as utilized to update therapy parameters before treatment. In another embodiment, the system may be configured to allow automated threshold measurements and updates, as enabled by the aforementioned Robotics sub-system, wherein the user may direct the robot, or the robot may be commanded to execute the measurements autonomously.

Software may also be configured, by working with computer processors and one or more function generators, amplifiers and therapy transducers, to allow various permutations of delivering and positioning optimized acoustic cavitation/histotripsy in and through a selected area/volume. This may include, but not limited to, systems configured with a fixed/natural focus arrangement using purely electromechanical positioning configuration(s), electronic beam steering (with or without electromechanical positioning), electronic beam steering to a new selected fixed focus with further electromechanical positioning, axial (Z axis) electronic beam steering with lateral (X and Y) electromechanical positioning, high speed axial electronic beam steering with lateral electromechanical positioning, high speed beam steering in 3D space, various combinations of including with dynamically varying one or more acoustic cavitation/histotripsy parameters based on the aforementioned ability to update treatment parameters based on threshold measurements (e.g., dynamically adjusting amplitude across the treatment area/volume).

### Other Components, Ancillaries and Accessories

The system may comprise various other components, ancillaries and accessories, including but not limited to computers, computer processors, power supplies including high voltage power supplies, controllers, cables, connectors, networking devices, software applications for security, communication, integration into information systems including hospital information systems, cellular communication devices and modems, handheld wired or wireless controllers, goggles or glasses for advanced visualization, augmented or virtual reality applications, cameras, sensors, tablets, smart devices, phones, internet of things enabling capabilities, specialized use “apps” or user training materials and applications (software or paper based), virtual proctors or trainers and/or other enabling features, devices, systems or applications, and/or methods of using the above.

### System Variations and Methods/Applications

In addition to performing a breadth of procedures, the system may allow additional benefits, such as enhanced planning, imaging and guidance to assist the user. In one embodiment, the system may allow a user to create a patient, target and application specific treatment plan, wherein the system may be configured to optimize treatment parameters based on feedback to the system during planning, and where planning may further comprise the ability to run various test protocols to gather specific inputs to the system and plan.

Feedback may include various energy, power, location, position, tissue and/or other parameters.

The system, and the above feedback, may also be further configured and used to autonomously (and robotically) execute the delivery of the optimized treatment plan and protocol, as visualized under real-time imaging during the procedure, allowing the user to directly observe the local treatment tissue effect, as it progresses through treatment, and start/stop/modify treatment at their discretion. Both test and treatment protocols may be updated over the course of the procedure at the direction of the user, or in some embodiments, based on logic embedded within the system.

It is also recognized that many of these benefits may further improve other forms of acoustic therapy, including thermal ablation with high intensity focused ultrasound (HIFU), high intensity therapeutic ultrasound (HITU) including boiling histotripsy (thermal cavitation), and are considered as part of this disclosure. The disclosure also considers the application of histotripsy as a means to activate previously delivered in active drug payloads whose activity is inert due to protection in a micelle, nanostructure or similar protective structure or through molecular arrangement that allows activation only when struck with acoustic energy.

In another aspect, the Therapy sub-system, comprising in part, one or more amplifiers, transducers and power supplies, may be configured to allow multiple acoustic cavitation and histotripsy driving capabilities, affording specific benefits based on application, method and/or patient specific use. These benefits may include, but are not limited to, the ability to better optimize and control treatment parameters, which may allow delivery of more energy, with more desirable thermal profiles, increased treatment speed and reduced procedure times, enable electronic beam steering and/or other features.

This disclosure also includes novel systems and concepts as related to systems and sub-systems comprising new and “universal” amplifiers, which may allow multiple driving approaches (e.g., single and multi-cycle pulsing). In some embodiments, this may include various novel features to further protect the system and user, in terms of electrical safety or other hazards (e.g., damage to transducer and/or amplifier circuitry).

In another aspect, the system, and Therapy sub-system, may include a plethora of therapy transducers, where said therapy transducers are configured for specific applications and uses and may accommodate treating over a wide range of working parameters (target size, depth, location, etc.) and may comprise a wide range of working specifications (detailed below). Transducers may further adapt, interface and connect to a robotically-enabled system, as well as the Coupling sub-system, allowing the transducer to be positioned within, or along with, an acoustic coupling device allowing, in many embodiments, concurrent imaging and histotripsy treatments through an acceptable acoustic window. The therapy transducer may also comprise an integrated imaging probe or localization sensors, capable of displaying and determining transducer position within the treatment site and affording a direct field of view (or representation of) the treatment site, and as the acoustic cavitation/histotripsy tissue effect and bubble cloud may or may not change in appearance and intensity, throughout the treatment, and as a function of its location within said treatment (e.g., tumor, healthy tissue surrounding, critical structures, adipose tissue, etc.).

The systems, methods and use of the system disclosed herein, may be beneficial to overcoming significant unmet needs in the areas of soft tissue ablation, oncology, immuno-oncology, advanced image guided procedures, surgical procedures including but not limited to open, laparoscopic, single incision, natural orifice, endoscopic, non-invasive, various combination of, various interventional spaces for catheter-based procedures of the vascular, cardiovascular pulmonary and/or neurocranial-related spaces, cosmetics/aesthetics, metabolic (e.g., type 2 diabetes), plastic and reconstructive, ocular and ophthalmology, orthopedic, gynecology and men's health, and other systems, devices and methods of treating diseased, injured, undesired, or healthy tissues, organs or cells.

Systems and methods are also provided for improving treatment patterns within tissue that can reduce treatment time, improve efficacy, and reduce the amount of energy and prefocal tissue heating delivered to patients.

### Use Environments

The disclosed system, methods of use, and use of the system, may be conducted in a plethora of environments and settings, with or without various support systems such as anesthesia, including but not limited to, procedure suites, operating rooms, hybrid rooms, in and out-patient settings, ambulatory settings, imaging centers, radiology, radiation therapy, oncology, surgical and/or any medical center, as well as physician offices, mobile healthcare centers or systems, automobiles and related vehicles (e.g., van), aero and marine transportation vehicles such as planes and ships, and/or any structure capable of providing temporary procedure support (e.g., tent). In some cases, systems and/or sub-systems disclosed herein may also be provided as integrated features into other environments, for example, the direct integration of the histotripsy Therapy sub-system into a MRI scanner or patient surface/bed, wherein at a minimum the therapy generator and transducer are integral to such, and in other cases wherein the histotripsy configuration further includes a robotic positioning system, which also may be integral to a scanner or bed centered design.

### Coupling

Systems may comprise a variety of Coupling sub-system embodiments, of which are enabled and configured to allow acoustic coupling to the patient to afford effective acoustic access for ultrasound visualization and acoustic cavitation/histotripsy (e.g., provide acoustic window and medium between the transducer(s) and patient, and support of). These may include different form factors of such, including open and enclosed device solutions, and some arrangements which may be configured to allow dynamic control over the acoustic medium (e.g., temperature, dissolved gas content, level of particulate filtration, sterility, volume, composition, etc.). Such dynamic control components may be directly integrated to the system (within the Cart), or may be in temporary/intermittent or continuous communication with the system, but externally situated in a separate device and/or cart.

The Coupling sub-system typically comprises, at a minimum, coupling medium (e.g., degassed water or water solutions), a reservoir/container to contain said coupling medium, and a support structure (including interfaces to other surfaces or devices). In most embodiments, the coupling medium is water, and wherein the water may be conditioned before or during the procedure (e.g., chilled, degassed, filtered, etc.). Various conditioning parameters may be employed based on the configuration of the system and its intended use/application.

The reservoir or medium container may be formed and shaped to various sizes and shapes, and to adapt/conform to the patient, allow the therapy transducer to engage/access and work within the acoustic medium, per defined and required working space (minimum volume of medium to allow the therapy transducer to be positioned and/or move through one or more treatment positions or patterns, and at various standoffs or depths from the patient, etc.), and wherein said reservoir or medium container may also mechanically support the load, and distribution of the load, through the use of a mechanical and/or electromechanical support structure. As a representative example, this may include a support frame. The container may be of various shapes, sizes, curvatures, and dimensions, and may be comprised of a variety of materials compositions (single, multiple, composites, etc.), of which may vary throughout. In some embodiments, it may comprise features such as films, drapes, membranes, bellows, etc. that may be insertable and removable, and/or fabricated within, of which may be used to conform to the patient and assist in confining/containing the medium within the container. It may further contain various sensors (e.g., volume/fill level), drains (e.g., inlet/outlet), lighting (e.g., LEDs), markings (e.g., fill lines, set up orientations, etc.), text (e.g., labeling), etc.

In one embodiment, the reservoir or medium container contains a sealable frame, of which a membrane and/or film may be positioned within, to afford a conformable means of contacting the reservoir (later comprising the treatment head/therapy transducer) as an interface to the patient, that further provides a barrier to the medium (e.g., water) between the patient and therapy transducer). In other embodiments, the membrane and/or film may comprise an opening, the patient contacting edge of which affords a fluid/mechanical seal to the patient, but in contrast allows medium communication directly with the patient (e.g., direct degassed water interface with patient). The superstructure of the reservoir or medium container in both these examples may further afford the proximal portion of the structure (e.g., top) to be open or enclosed (e.g., to prevent spillage or afford additional features).

Disclosed membranes may be comprised of various elastomers, viscoelastic polymers, thermoplastics, thermoplastic elastomers, thermoset polymers, silicones, urethanes, rigid/flexible co-polymers, block co-polymers, random block co-polymers, etc. Materials may be hydrophilic, hydrophobic, surface modified, coated, extracted, etc., and may also contain various additives to enhance performance, appearance or stability. In some embodiments, the thermoplastic elastomer may be styrene-ethylene-butylene-styrene (SEBS), or other like strong and flexible elastomers. The membrane form factor can be flat or pre-shaped prior to use. In other embodiments, the membrane could be inelastic (i.e., a convex shape) and pressed against the patient's skin to acoustically couple the transducer to the tissue. Systems and methods are further disclosed to control the level of contaminants (e.g., particulates, etc.) on the membrane to maintain the proper level of ultrasound coupling. Too many particulates or contaminants can cause scattering of the ultrasound waves. This can be achieved with removable films or coatings on the outer surfaces of the membrane to protect against contamination.

Said materials may be formed into useful membranes through molding, casting, spraying, ultrasonic spraying, extruding, and/or any other processing methodology that produces useful embodiments. They may be single use or reposable/reusable. They may be provided non-sterile, aseptically cleaned or sterile, where sterilization may comprise any known method, including but not limited to ethylene oxide, gamma, e-beam, autoclaving, steam, peroxide, plasma, chemical, etc. Membranes can be further configured with an outer molded or over molded frame to provide mechanical stability to the membrane during handling including assembly, set up and take down of the coupling sub-system. Various parameters of the membrane can be optimized for this method of use, including thickness, thickness profile, density, formulation (e.g., polymer molecular weight and copolymer ratios, additives, plasticizers, etc.), including optimizing specifically to maximize acoustic transmission properties, including minimizing impact to cavitation initiation threshold values, and/or ultrasound imaging artifacts, including but not limited to membrane reflections, as representative examples.

Open reservoirs or medium containers may comprise various methods of filling, including using pre-prepared medium or water, that may be delivered into the containers, in some cases to a defined specification of water (level of temperature, gas saturation, etc.), or they may comprise additional features integral to the design that allow filling and draining (e.g., ports, valves, hoses, tubing, fittings, bags, pumps, etc.). These features may be further configured into or to interface to other devices, including for example, a fluidics system. In some cases, the fluidics system may be an in-house medium preparation system in a hospital or care setting room, or conversely, a mobile cart-based system which can prepare and transport medium to and from the cart to the medium container, etc.

Enclosed iterations of the reservoir or medium container may comprise various features for sealing, in some embodiments sealing to a proximal/top portion or structure of a reservoir/container, or in other cases where sealing may comprise embodiments that seal to the transducer, or a feature on the transducer housings. Further, some embodiments may comprise the dynamic ability to control the volume of fluid within these designs, to minimize the potential for air bubbles or turbulence in said fluid and to allow for changes in the focal length to the target area without moving the transducer. As such, integrated features allowing fluid communication, and control of, may be provided (ability to provide/remove fluid on demand), including the ability to monitor and control various fluid parameters, some disclosed above. In order to provide this functionality, the overall system, and as part, the Coupling sub-system, may comprise a fluid conditioning system, which may contain various electromechanical devices, systems, power, sensing, computing, pumping, filtering and control systems, etc. The reservoir may also be configured to receive signals that cause it to deform or change shape in a specific and controlled manner to allow the target point to be adjusted without moving the transducer.

Coupling support systems may include various mechanical support devices to interface the reservoir/container and medium to the patient, and the workspace (e.g., bed, floor, etc.). In some embodiments, the support system comprises a mechanical arm with 3 or more degrees of freedom. Said arm may have a proximal interface with one or more locations (and features) of the bed, including but not limited to, the frame, rails, customized rails or inserts, as well as one or more distal locations of the reservoir or container. The arm may also be a feature implemented on one or more Carts, wherein Carts may be configured in various unlimited permutations, in some cases where a Cart only comprises the role of supporting and providing the disclosed support structure.

In some embodiments, the support structure and arm may be a robotically-enabled arm, implemented as a stand-alone Cart, or integrated into a Cart further comprising two or more system sub-systems, or where in the robotically-enabled arm is an arm of another robot, of interventional, surgical or other type, and may further comprise various user input features to actuate/control the robotic arm (e.g., positioning into/within coupling medium) and/or

Coupling solution features (e.g., filling, draining, etc.). In some examples, the support structure robotic arm positional encoders may be used to coordinate the manipulation of the second arm (e.g. comprising the therapy transducer/treatment head), such as to position the therapy transducer to a desired/known location and pose within the coupling support structure.

Overall, significant unmet needs exist in interventional and surgical medical procedures today, including those procedures utilizing minimally invasive devices and approaches to treat disease and/or injury, and across various types of procedures where the unmet needs may be solved with entirely new medical procedures. Today's medical system capabilities are often limited by access, wherein a less or non-invasive approach would be preferred, or wherein today's tools aren't capable to deliver preferred/required tissue effects (e.g., operate around/through critical structures without serious injury), or where the physical set up of the systems makes certain procedure approaches less desirable or not possible, and where a combination of approaches, along with enhanced tissue effecting treatments, may enable entirely new procedures and approaches, not possible today.

In addition, specific needs exist for enabling histotripsy delivery, including robotic histotripsy delivery, wherein one or more histotripsy therapy transducers may be configured to acoustically couple to a patient, using a completely sealed approach (e.g., no acoustic medium communication with the patient's skin) and allowing the one or more histotripsy transducers to be moved within the coupling solution without impeding the motion/movement of the robotic arm or interfering/disturbing the coupling interface, which could affect the intended treatment and/or target location.

Disclosed herein are histotripsy acoustic and patient coupling systems and methods, to enable histotripsy therapy/treatment, as envisioned in any setting, from interventional suite, operating room, hybrid suites, imaging centers, medical centers, office settings, mobile treatment centers, and/or others, as non-limiting examples. The following disclosure further describes novel systems used to create, control, maintain, modify/enhance, monitor and setup/takedown acoustic and patient coupling systems, in a variety of approaches, methods, environments, architectures and work-flows. In general, the disclosed novel systems may allow for a coupling medium, in some examples degassed water, to be interfaced between a histotripsy therapy transducer and a patient, wherein the acoustic medium provides sufficient acoustic coupling to said patient, allowing the delivery of histotripsy pulses through a user desired treatment location (and volume), where the delivery may require physically moving the histotripsy therapy transducer within a defined work-space comprising the coupling medium, and also where the coupling system is configured to allow said movement of the therapy transducer (and positioning system, e.g., robot) freely and unencumbered from by the coupling support system (e.g., a frame or manifold holding the coupling medium).

### Coupling System and Sub-Systems/Components

The disclosed histotripsy acoustic and patient coupling systems, in general, may comprise one or more of the following sub-systems and components, including but not limited to 1) a membrane/barrier film to provide an enclosed, sealed and conformal patient coupling and histotripsy system interface, 2) a frame and assembly to retain the membrane and provide sufficient work and head space for a histotripsy therapy transducers required range of motion (x, y and z, pitch, roll and yaw), 3) a sufficient volume of ultrasound medium to afford acoustic coupling and interfaces to a histotripsy therapy transducer and robotic arm, 4) one or more mechanical support arms to allow placement, positioning and load support of the frame, assembly and medium and 5) a fluidics system to prepare, provide and remove ultrasound medium(s) from the frame and assembly.

In some embodiments, the coupling system may be fully sealed, and in other embodiments and configurations, it may be partially open to afford immediate access (physical and/or visual).

The acoustic and patient coupling systems and sub-systems may further comprise various features and functionality, and associated work-flows, and may also be configured in a variety of ways to enable histotripsy procedures as detailed below.

The disclosed histotripsy acoustic and patient coupling systems, in general, may comprise one or more of the following sub-systems and components, including but not limited to 1) a membrane/barrier film to provide an enclosed, sealed and conformal patient coupling and histotripsy system interface, 2) a frame and assembly to retain the membrane and provide sufficient work and head space for a histotripsy therapy transducers required range of motion (x, y and z, pitch, roll and yaw), 3) a sufficient volume of ultrasound medium to afford acoustic coupling and interfaces to a histotripsy therapy transducer and robotic arm, 4) one or more mechanical support arms to allow placement, positioning and load support of the frame, assembly and medium and 5) a fluidics system to prepare, provide and remove ultrasound medium(s) from the frame and assembly.

In some embodiments, the coupling system may be fully sealed, and in other embodiments and configurations, it may be partially open to afford immediate access (physical and/or visual).

The acoustic and patient coupling systems and sub-systems may further comprise various features and functionality, and associated work-flows, and may also be configured in a variety of ways to enable histotripsy procedures as detailed below.

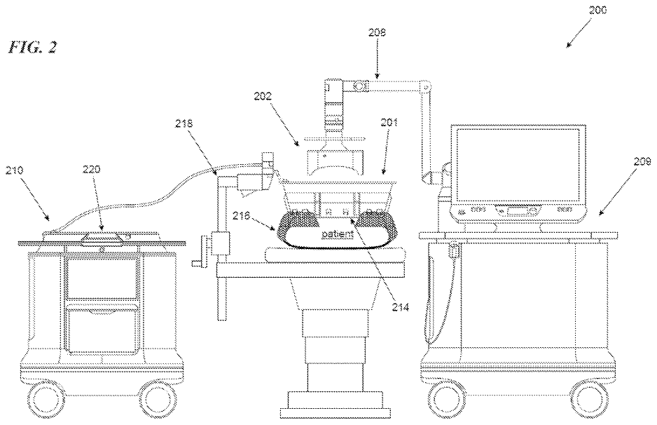

FIG. 2 illustrates one embodiment of a histotripsy therapy and imaging system 200, including a coupling assembly 201. As described above, a histotripsy therapy and imaging system can include a therapy transducer 202, an imaging system, a robotic positioning arm 208, and a fluidics cart 210. The robotic positioning arm may be attached to a therapy cart, such as cart 209.

The therapy and/or imaging transducers can be disposed within the coupling assembly 201 which can further include a coupling membrane 214 and a membrane constraint 216 configured to prevent the membrane from expanding too far from the transducer. The coupling membrane can be filled with an acoustic coupling medium such as a fluid or a gel. The membrane constraint can be, for example, a semi-rigid or rigid material as compared to the membrane, and configured to restrict expansion/movement of the membrane. In some embodiments, the membrane constraint is not used, and the elasticity and tensile strength of the membrane prevent over expansion. The coupling membrane can be a mineral-oil infused SEBS membrane to prevent direct fluid contact with the patient's skin. In the illustrated embodiment, the coupling assembly 201 is supported by a mechanical support arm 218 which can be load bearing in the x-y plane but allow for manual or automated z-axis adjustment. The mechanical support arm can be attached to the floor, the patient table, or the fluidics cart 210. The mechanical support is designed and configured to conform and hold the coupling membrane 214 in place against the patient's skin while still allowing movement of the therapy/imaging transducer relative to the patient and also relative to the coupling membrane 214 with the robotic positioning arm 208.

The fluidics cart 210 can include additional features, including a fluid tank 220, a cooling and degassing system, and a programmable control system. The fluidics cart is configured for external loading of the coupling membrane with automated control of fluidic sequences. Further details on the fluidics cart are provided below.

The therapy and/or imaging transducers can be housed in a coupling assembly which can further include a coupling membrane and a membrane constraint configured to prevent the membrane from expanding too far from the transducer. The coupling membrane can be filled with an acoustic coupling medium such as a fluid or a gel. The membrane constraint can be, for example, a semi-rigid or rigid material configured to restrict expansion/movement of the membrane. In some embodiments, the membrane constraint is not used, and the elasticity and tensile strength of the membrane prevent over expansion. The coupling membrane can be a mineral-oil infused SEBS membrane to prevent direct fluid contact with the patient's skin. In the illustrated embodiment, the coupling assembly is supported by a mechanical support arm which can be load bearing in the x-y plane but allow for manual or automated z-axis adjustment. The mechanical support arm can be attached to the floor, the patient table, or the cart. The mechanical support is designed and configured to conform and hold the coupling membrane in place against the patient's skin while still allowing movement of the therapy/imaging transducer relative to the patient and also relative to the coupling membrane with the robotic positioning arm.

The system can further include a fluidics system that can include a fluid source, a cooling and degassing system, and a programmable control system. The fluidics system is configured for external loading of the coupling membrane with automated control of fluidic sequences. Further details on the fluidics system are provided below.

### Membranes/Barrier Films and Related Architectures

Membranes and barrier films may be composed of various biocompatible materials which allow conformal coupling to patient anatomy with minimal or no entrapped bubbles capable of interfering with ultrasound imaging and histotripsy therapy, and that are capable of providing a sealed barrier layer between said patient anatomy and the ultrasound medium, of which is contained within the work-space provided by the frame and assembly.

Membrane and barrier film materials may comprise flexible and elastomeric biocompatible materials/polymers, such as various thermoplastic and thermoset materials, as well as permanent or bioresorbable polymers. Additionally, the frame of the UMC can also comprise the same materials. In some examples, the membrane may be rigid or semi-rigid polymers which are pre-shaped or flat.

### Ultrasound Medium

As previously described, the ultrasound medium may comprise any applicable medium capable of providing sufficient and useful acoustic coupling to allow histotripsy treatments and enable sufficient clinical imaging (e.g., ultrasound). Ultrasound mediums, as a part of this disclosure and system, may comprise, but are not limited to, various aqueous solutions/mediums, including mixtures with other co-soluble fluids, of which may have preferred or more preferred acoustic qualities, including ability to match speed of sound, etc. Example mediums may comprise degassed water and/or mixtures/co-solutions of degassed water and various alcohols, such as ethanol.

### Mechanical Support Arms and Arm Architectures

In order to support the acoustic and patient coupling system, including providing efficient and ergonomic work-flows for users, various designs and configurations of mechanical support arms (and arm architectures) may be employed. Support arms may be configured with a range of degrees of freedom, including but not limited to allowing, x, y, z, pitch, roll and yaw, as well additional interfacing features that may allow additional height adjustment or translation.

Arms may comprise a varied number and type of joints and segments. Typically, arms may comprise a minimum of 2 segments. In some configurations, arms may comprise 3 to 5 segments.

Arms are also be configured to interface proximally to a main support base or base interface (e.g., robot, table, table/bed rail, cart, floor mount, etc.) and distally to the frame/assembly and overall “UMC” or “coupling solution”. This specific distal interface may further include features for controlling position/orientation of the frame/assembly, at the frame/assembly interface.

For example, in some embodiments, the arm/frame interface may comprise a ball joint wrist. In another example, the interface may include use of a gimbal wrist or an adjustable pitch and roll controlled wrist. These interfaces may be further employed with specific user interfaces and inputs, to assist with interacting with the various wrists, of which may include additional handles or knobs (as an unlimited example), to further enable positioning the UMC/coupling solution. For example, a gimbal wrist may benefit from allowing the frame/assembly to have 3 degrees of freedom (independent of the arm degrees of freedom), including pitch, roll and yaw adjustments.

Support arms, configured with arm wrists, further interfaced with frames/assemblies, may comprise features such as brakes, including cable or electronic actuated brakes, and quick releases, which may interact with one or more axis, individually, or in groupings. They may also include electronic lift systems and base supports. In some embodiments, these lift systems/base supports are co-located with robot arm bases, wherein said robot arm is equipped with the histotripsy therapy transducer configured to fit/work within the enclosed coupling solution. In other embodiments, the support arm is located on a separate cart. In some cases, the separate cart may comprise a fluidics system or user console. In other embodiments, it is interfaced to a bed/table, including but not limited to a rail, side surface, and/or bed/table base. In other examples/embodiments, it's interfaced to a floor-based structure/footing, capable of managing weight and tipping requirements.

### Fluidics Systems, Control Systems and System Architectures

As a part of overall fluidics management, histotripsy systems including acoustic/patient coupling systems, may be configured to include an automated fluidics system, which primarily is responsible for providing a reservoir for preparation and use of coupling medium, where preparation may include the ability to degas, chill, monitor, adjust, dispense/fill, and retrieve/drain coupling medium to/from the frame/assembly. The fluidics system may include an emergency high flow rate system for rapid draining of the coupling medium from the UMC. In some embodiments, the fluidics system can be configured for a single use of the coupling medium, or alternatively, for re-use of the medium. In some embodiments, the fluidics system can implement positive air pressure or vacuum to carry out leak tests of the UMC and membrane prior to filling with a coupling medium. Vacuum assist can also be used for removal of air from the UMC during the filling process. The fluidics system can further include filters configured to prevent particulate contamination from reaching the UMC.

The fluidics system may be implemented in the form of a mobile fluidics cart. The cart may comprise an input tank, drain tank, degassing module, fill pump, drain pump, inert gas tank, air compressor, tubing/connectors/lines, electronic and manual controls systems and input devices, power supplies and one or more batteries. The cart in some cases may also comprise a system check vessel/reservoir for evaluating histotripsy system performance and related system diagnostics (configured to accommodate a required water volume and work-space for a therapy transducer).

### Integrated MR and US Guided Histotripsy Systems and Methods

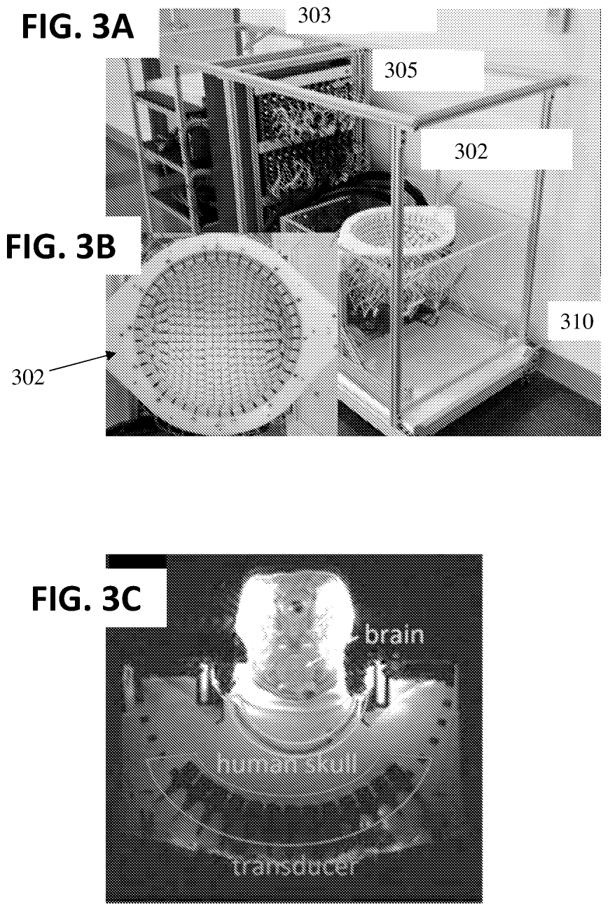

Referring to FIGS. 3A-3B, a transcranial histotripsy array 302 is provided that is configured to treat a wide range of brain locations in diverse patients. The histotripsy array is designed substantially smaller element size and higher power output than prior systems by leveraging three recent technology advancements. 1) New piezoelectric material configured to produce a 2.5 MPa surface pressure, compared to the 1.5 MPa of prior arrays, providing the potential to increase transducer output by 67%, 2) Improved rapid prototyping array manufacturing method using arbitrary shaped elements can increase the packing density from 74% (prior arrays) to >90%, and 3) Reducing element size can improve aberration correction. By improving the transducer material, increasing the packing density, and reducing element size, this new transcranial histotripsy array of the present disclosure can provide triple the power output of prior arrays. These developments are essential to enable transcranial treatment of a wide range of brain locations in diverse patients.

FIG. 3A shows additional components in a test system, including power supply 303, transmit-receive driving electronics 305, and a cart 310. Any of these systems can be incorporated into a commercial system such as the histotripsy therapy system 100 shown in FIG. 1A. FIG. 3C shows the transducer array positioned adjacent to a human skull and targeting brain tissue of the subject.

This disclosure provides novel integrated transcranial magnetic resonance (MR) and ultrasound (US) guided histotripsy (tcMfR-USgHt) systems that further improve the treatment guidance, safety, and accuracy of histotripsy. The systems provided herein can provide pre-treatment targeting using MR-thermometry and low temperature heating generated by the histotripsy array. MRI can also be used to visualize histotripsy-generated damage in the brain (dMRI) and blood products. However, the signal-to-noise ratio (SNR) of the MRI signals is typically limited due to suboptimal RF coils, and MRI cannot provide high temporal resolution (≥50 Hz) at the rate that histotripsy pulses are delivered. In addition, MRI cannot be used to detect pre-focal cavitation at high-pressure hotspots near or on the skull surface.

In one embodiment, the transducer array 302 array integrates a specialized RF surface coil (e.g., the AIR™ coil from GE Healthcare) with minimal ultrasound attenuation (<5%) into the transducer design. The coils can be integrated into the transducer, or alternatively positioned near the transducer or around the patient, to allow for transmitting and receiving of ultrasound pulses (and received ACE signals) with the transducer array while also facilitating receiving MR signals with the AIR coils. These coils can substantially increase the SNR (13×) of received MRI signals. In addition, a transmit-receive electrical circuitry design can be coupled to the transducer array to generate histotripsy while enabling the same array to detect acoustic cavitation emission (ACE) signals with high sensitivity and dynamic range. This transmit-receive driver can be used for cavitation mapping and damage monitoring in the brain including locations on the skull surface and at a frame rate ≥50 Hz. The tcMR-USgHt system of this disclosure can integrate a custom AIR coil for improved MRI guidance and real-time US cavitation mapping at all intracranial locations and at a high frame rate realistic for monitoring histotripsy delivery.

The tcMRgHt system shown in FIGS. 3A-3C can include a 360-element, hemispherical phased-array (fc=700 kHz, focal depth=15 cm). In some examples, this histotripsy system is configured to generate an estimated peak negative pressure up to 300 MPa. The focal zone can be approximately 1.2×1.2×2.3 mm in free field, with a −6 dB electronic focal steering range of 33 mm laterally and 50 mm axially. A highly effective and fast 2-step aberration correction (AC) method can correct the acoustic aberration through the skull based on prior CT scans of the patient's head co-registered to the MR images, followed by a cavitation-based approach using the ACE signals. Results show that the 2-step AC achieves 90±7% peak focal pressure compared to the gold standard hydrophone correction.

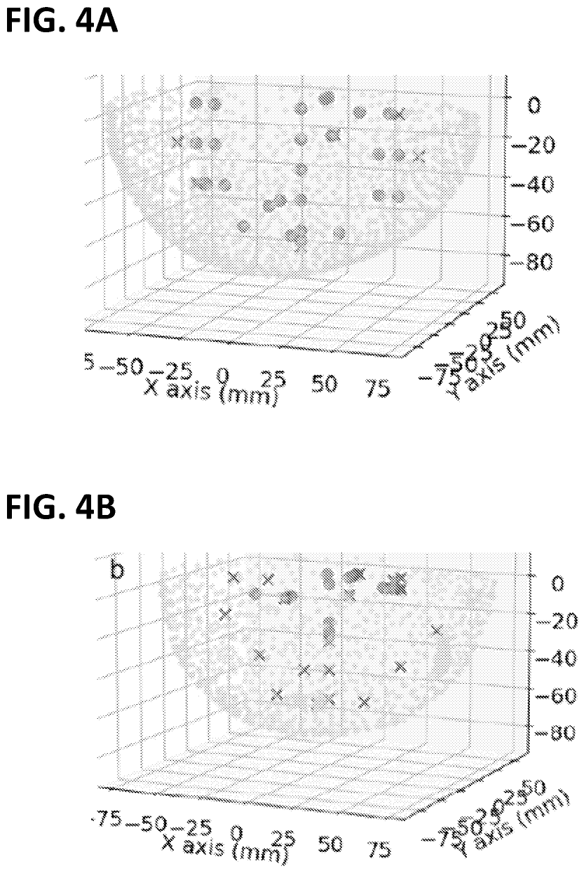

The tcMRgHt system can be used to treat a wide range of locations through skulls with a high SDR (low density variation) or small thickness, while the treatment location envelop is limited to close to the central regions through skulls with a low SDR or large thickness. For example, in experiments, Skull 1 (SDR=0.7; average thickness=4.9 mm), experimental results show that the tcMRgHt system was able to generate cavitation confirmed by ACE mapping at locations from deep to within 1 cm from the skull interior surface in superior, anterior, posterior, and lateral regions (FIG. 4A). In comparison, in Skull 2 (SDR=0.3; average thickness=6.5 mm), cavitation could only be generated up to 3.5 cm from the skull surface (FIG. 4B). Simulations performed on CT scans of seven skulls show that skulls with a lower SDR and higher average thickness result in a higher acoustic attenuation and more pre-focal pressure hotspots, which prevent effective treatment (more details Table 1). Simulation shows that treatment location limitation can be overcome by a new histotripsy array with substantially increased power and reduced element size.

TABLE 1
Acoustic Properties and Simulation Results of 7 Skulls

| | | Mean | | Attenuation at | Attenuation at | Pre-focal hotspot |
|---|---|---|---|---|---|---|
| Skull | Dimension | thickness | | central target | 1 cm to skull | for target 1 cm to |
| No. | (mm) | (mm) | SDR | with AC | surface with AC | skull surface? |
| 1 | 179 × 142 × 155 | 4.9 | 0.71 | 0.63 | 0.88-0.93 | Yes |
| 2 | 177 × 139 × 172 | 6.5 | 0.35 | 0.73 | 0.90-0.94 | No |
| 3 | 185 × 142 × 194 | 8.7 | 0.48 | 0.73 | 0.87-0.90 | Yes |
| 4 | 193 × 144 × 201 | 6.8 | 0.48 | 0.70 | 0.86-0.93 | No |
| 5 | 209 × 146 × 160 | 7.0 | 0.59 | 0.60 | 0.85-0.90 | No |
| 6 | 184 × 130 × 151 | 7.0 | 0.71 | 0.74 | 0.87-0.93 | Yes |
| 7 | 182 × 149 × 157 | 7.1 | 0.51 | 0.69 | 0.85-0.93 | No |

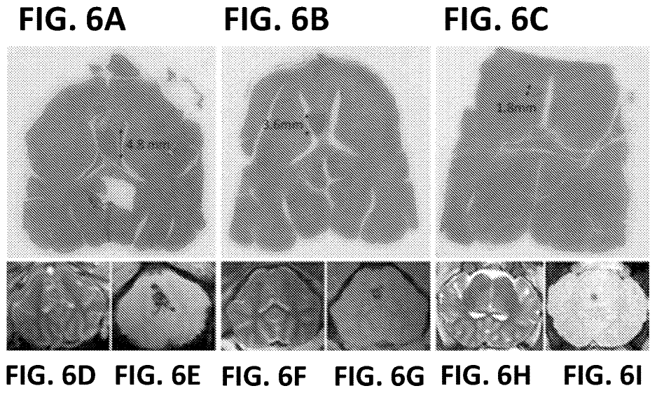

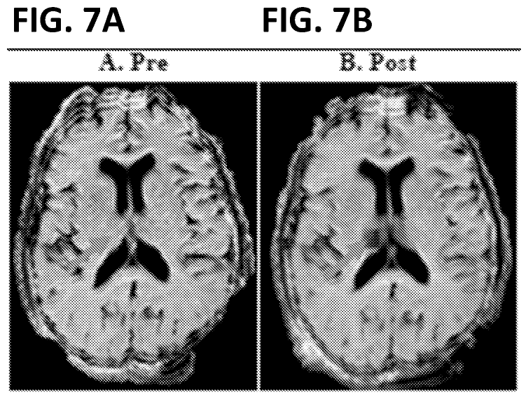

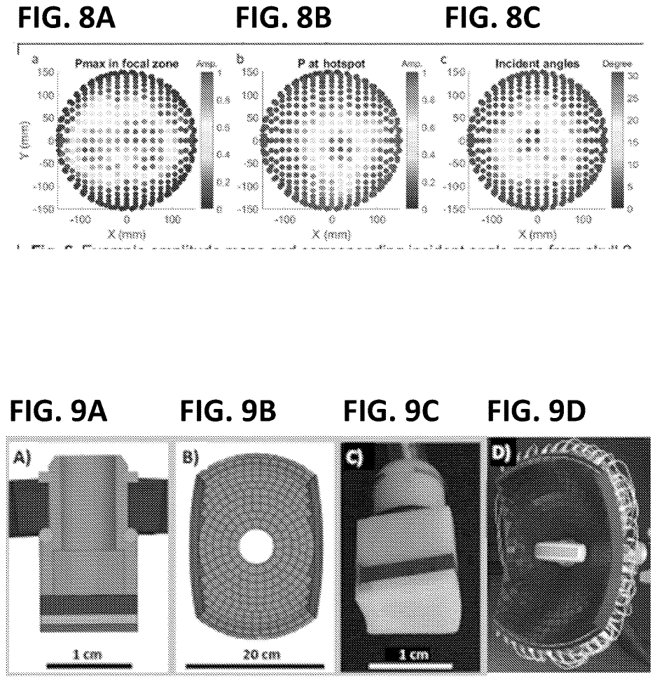

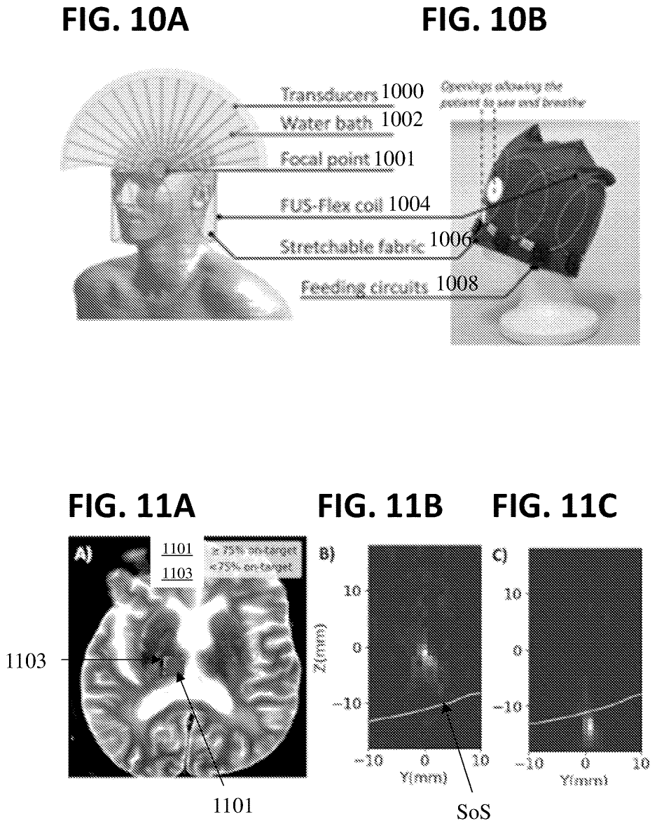

This disclosure investigates MR thermometry for pre-treatment targeting of tcMRgHt, where driving electronics of the transcranial histotripsy array are configured to enable low temperature heating. For example, in some embodiments, the pulse sequence is modified from a typical non-thermal histotripsy sequence to one that generates low heating in tissue (e.g., 1-2 deg C). It should be noted that the temperature increase is not sufficient to produce HIFU thermal ablation, but does provide enough heat to be measured with MR thermometry. FIG. 5A shows a region of tissue heating surrounding the focal zone FZ. While histotripsy is typically considered a non-thermal therapy in which no tissue heating is produced around a focal zone, the driving electronics and/or pulse sequences of the present disclosure can be modified to enable small (e.g., 1-2 deg C. or less) heating in tissue surrounding the focal zone. The targeting accuracy of using MR-thermometry and low-heating for histotripsy can be measured by the distance between the center of heating map on pre-treatment MR-thermometry and the center of the histotripsy lesion on post-treatment DWI MRI (dMRI), showing the MR-thermometry focus MRTF and geometric focus GF (FIG. 5B). In other embodiments, the center of the heating map can be compared to the expected focal location to indicate if the actual focal location (or cavitation, as indicated by the heating) is positioned at the expected focus. In one experiment, Histotripsy-generated damage in ex vivo brain was only visible on dMRI (FIG. 5C). As histotripsy mechanically breaks down the cellular members and extracellular matrix, the apparent diffusion coefficient (ADC) in the tissue changes to show on dMRI immediately post treatment. There is a trend of increasing AADC with increasing histotripsy doses (FIG. 5D). In vivo, due to bleeding inside the histotripsy lesions, they are also evident on T2*, T2, T1-weighted MR images (FIGS. 6A-6I) immediately after treatment.

The feasibility and performance of the tcMRgHt system was experimentally demonstrated in the in vivo porcine brain through an excised human skull and in the brain of full human cadavers. TcMRgHt treatment was delivered to the in vivo intact brain of 8 pigs through an excised human skull. After craniectomy to open an acoustic window to the brain, the skin was sutured over the intact dura. Two days after the craniectomy, histotripsy was applied to the intact pig brain through an excised human calvarium covering the craniectomy defect. Histotripsy pulses were delivered at a pulse repetition frequency (PRF) of 10 Hz to 3 ×3×3 mm³ or 6×6 ×6 mm³ grids with 1 or 1.5 mm grid spacing and 50 pulses/location at a peak negative pressure (p−) of ~48 MPa. MRI showed successful histotripsy ablation in all 8 pigs. MRI-evident lesions were well-confined within the targeted volume, without excessive bleeding or edema outside of the target zone. Histology revealed cellular disruption within the ablation zones with sharp demarcation between treated and untreated tissue, which correlated well with the treatment zones on MRI (FIGS. 6A-6I). Shrunken neurons were noted within 500 μm around the ablation zone, while further distant neurons remained intact and normal neutrophils were also observed around the ablation zone.

In another experiment, evaluation of transcranial histotripsy in the brain of two full human cadavers was conducted within 96 hours postmortem. The cadaver heads were imaged with CT and MRI before treatment. The 2-step aberration correction was performed. Lesions of 1 cm³ was created by electronically steering the focus with a 1.1 mm spacing using 1-cycle pulses, 200 Hz PRF, p− of ~30 MPa, and 50 pulses per focal location. After treatment, the brain was imaged with MRI and extracted for histology. Three lesions were generated in the septum, corpus collosum, and thalamus in two cadaver brains. Representative pre- and post-treatment dMRI images of one lesion are shown in FIGS. 7A-7B.

In designing a transcranial ultrasound system, ultrasound propagation simulations can be performed on a number of CT head scans of patients covering diverse skull densities, thicknesses, and sizes to calculate the attenuation and pre-focal pressure hotspots for different target locations within the skull. Based on the calculations above, a histotripsy array was designed to achieve the focal pressure through the skull to exceed the cavitation intrinsic threshold at a wide range of brain locations (from deep locations to within 1 cm from the skull surface) and reduce pre-focal hotspots in diverse (>70%) patients. The new transcranial array, with associated transmit-receive capable driving electronics, can be constructed using a rapid prototyping approach. The transcranial array can be integrated with a MR-compatible positioner, stereotactic frame, and acoustic coupling to form a complete system.

Full wave propagation simulation was performed based on CT scans of seven skulls with a range of SDR's (0.3-0.7), dimensions, and thicknesses (4.9-8.7 mm) (Table 1), using K-wave and the 700 kHz, 360-element, 30-cm diameter hemispherical array. The acoustic attenuation at different locations within each skull was simulated to range 63-74% in the center location in the skull. When the array focus was placed closer to the skull surface, the attenuation increased, resulting in 80-94% at 1 cm from the skull surface. The simulation results were validated with pressure measurements and cavitation detection for Skull 1 and 2. Pre-focal hotspots—where pressure is higher than at the focus—exist mostly on the skull surface or within the skull and can occur when targeting near the skull surface. Simulation shows that reduced element size (i.e., more elements) significantly reduces the hotspot pressure by improving aberration correction. For example, for Skull 2 when focusing at a superior target 1 cm from the skull interior surface, using 1063 elements reduced the pressure at the pre-focal hotspot by 44% compared to using the 360-element array. Simulation shows that majority of the hotspot pressure is also attributed to a small number of elements, e.g., in Skull 2, 50% of the pressure in the hotspots are attributed to 16% of the array elements (FIGS. 8A-8C). Thus, the hotspot issue may be overcome through better aberration correction and/or running these elements at reduced amplitude or even turning off these elements. These preliminary results indicate the treatment location envelop limitation using prior histotripsy arrays in skulls with a mid to low SDR or large thicknesses. A new histotripsy array according to this disclosure with significantly increased pressure output and reduced element size can overcome the high attenuation and hotspot problems to expand the treatment location envelop for diverse patients.

Histotripsy Array Construction-a previously developed rapid prototyping method for ultrasound transducer construction can be used to build the current histotripsy array. According to one embodiment, the piezoelectric (PZT) elements can be placed into an individual module housing. The modules can then be assembled to a hemispherical scaffold. The array module housing and the scaffold of the entire array can be designed, for example, in Solidworks and fabricated using stereolithography (SLA) with a proprietary photopolymer selected based on the acoustic properties. Transducers built with this approach are low cost, MRI-compatible, and each individual element is replaceable. In one embodiment, the 360-element array uses identical square-shaped elements to simplify construction, limiting the packing density (74%, total PZT surface area/divided by the transducer total surface area) and maximum power output. The array construction technique can be improved to increase the packing density through arbitrarily shaped elements and thin electrically insulating epoxy coatings instead of separate module housings to maximize aperture utilization. This new method was used to build a 750 kHz truncated circular aperture transducer segmented into 260 arc-shaped modular elements (FIGS. 9A-9D, with FIGS. 9A and 9C showing the individual elements and FIGS. 9B and 9D showing the completed array). Individual PZT elements can be water jet cut, bonded to the matching layers, electrically insulated by 125 μm thick epoxy coatings, and assembled into a machined scaffold. This 260-element array achieved a packing density of 92%. This new method maximizes the packing density, allows replacement of individual transducer modules, and significantly reduces the fabrication cost for a large aperture, low f-number transducer arrays compared to conventional manufacturing approaches.

An innovative transmit-receive electrical circuitry design has been developed with high sensitivity and dynamic range to detect acoustic emissions from cavitation with the same array capable of transmitting electrical bursts up to several thousand volts for histotripsy. Full transmit-receive circuitry has been implemented on the current 360-channel array system enabling real-time 3D cavitation mapping and damage evaluation induced by histotripsy. The array has full transmit-receive capability on each element similar to an ultrasound imaging system. A novel “current sensing” approach is used for detection in place of a typical transmit-receive switch which would be impractical to implement due to the extremely high transmit voltages required for histotripsy. Standard integrated circuit digitizers designed for ultrasound imaging are used by the system to receive acoustic echoes and include a variable gain amplifier for wide dynamic range and high sensitivity. The incoming data stream on receive can be handled by 45 multicore ARM processor SOC/FPGAs. These have the capability for significant real-time parallel data processing.

In one experiment, the acoustic attenuation and pre-focal hotspots can be calculated for a wide range of treatment locations through skulls with different properties based on the patient CT head scans. Full wave acoustic propagation can be performed through the skull based on human patient CT head scans in K-wave on the Great Lakes compute cluster. Scans can be collected that will cover diverse skull density variation (SDR=0.2-0.8), skull thicknesses (average=4-8 mm), and dimensions. The speed of sound over the skull will be allowed to vary based on the analysis of the Hounsfield unit from the CT Bone+ scan instead of using a uniform speed. A 30 cm diameter hemispherical array geometry and a 700 kHz center frequency with different element numbers (up to 4096) were experimentally tested. Next, the ultrasound attenuation and pressure map (including pre-focal hotspots) are calculated through the skull as a function of target locations (deep to close to anterior, posterior, superior, and lateral skull surface). An optimization routine is then used to adjust the output of elements based on their relative contributions to pressure at the desired focal location vs any hotspots. The target peak pressure for the hotspot will be set to be less than 50% of the desired focal location while still maximizing the focal pressure.

Based on the attenuation range above, the new array has increased power output, with the goal of generating sufficient focal pressure (>30 MPa, above cavitation intrinsic threshold) through the skull to treat a wide range of locations (from the base to <1 cm of the skull surface) for >70% patients. To increase the array pressure output, the element size can be reduced, the packing density can be increased, and the PZT material can be improved.

First, the array element size must be determined. Reducing the element size can improve aberration correction, thus increasing the achievable focal pressure. Simulation can be performed to examine the focal pressure recovery after aberration correction as a function of the element size. Initial simulation shows increasing the element number to 1063 (reducing element size from 9 mm to 6 mm) results in 47% increase of focal pressure. The upper limit of the array element number is set to be 4096 due to the electric driver complexities. Second, the new array mechanical design can be performed in SolidWorks, with the goal to achieve a packing density >90%. In one example, the array uses ~100% packing with equal area polygons and a near ideal pseudorandom arrangement. The pseudorandom element arrangement minimizes side lobes and improves electric focal steering. Third, the array can use a different PZT material. The prior transcranial array uses a porous hard PZT material (PZ36, Meggitt) with a low acoustic impedance. Soft PZTs (PZT5-2 MPa) and 1-3 PZT composites (Imasonic-2.5 MPa) both can produce higher surface pressure compared to the PZ36 (1.5 MPa). The maximal focal pressure can be calculated based on the PZT surface pressure, packing density, and the attenuation calculated above using the increased element number. With these three improvements, the new array can increase the pressure output by 204% (Table 2), more than triple the output of the existing array.

TABLE 2
Estimated Pressure Increase of the New Array

| | Packing | PZT | Aberration | Total |
|---|---|---|---|---|
| | Density | material | Correction | Increase |
| Pressure | 24% | 67% | 47% | 204% |
| Increase | | | | |

The histotripsy array of the present disclosure can be constructed based on the design above using the new rapid prototyping method as described above, which allows us to achieve a packing density even higher than conventional transducer manufacturing method due to small spacing (125 μm) between elements enabled by high dielectric strength of the insulating coatings. The transmit-receive capable driving electronics can be constructed as described above. The histotripsy array adheres to standards for a MR conditional device. All metallic components (fasteners, cables, etc.) can comprise of non-ferrous materials. This design, using polyaramid housings and 3D printed matching layers, minimizes the mass of metal. The modular construction technique used for this system automatically results in a segmented ground plane. Segmented ground planes have been shown to yield much better MR image quality compared to continuous plane designs, allowing RF magnetic field penetration through spaces between the ground plane segments. Four small MRI fiducial markers can be affixed to the array scaffold to simplify co-registration between the ultrasound and MRI coordinate spaces-a standard technique used in with tcMRgFUS systems.

In addition to the histotripsy array, the following components can form the integrated tcMR-USgHt system: 1) a MR-conditional mechanical positioner with translation and rotation mobility to move the histotripsy array, 2) a stereotactic frame to rigidly fix the patient head and facilitate aberration correction, 3) an acoustic coupler to ensure the ultrasound transmission from the array to the head, and 4) a support structure to rigidly support all the components.

1) A MR-compatible mechanical positioner. The positioner can have ±10 cm movement range with 0.2 mm step size and rotation movement (1.8° step angle), which in combination with the electric focal steering capability of the array allows the system align and scan over brain tumors of any size and location. One example of a robotic arm suitable for such use is shown in FIG. 1A.

2) A MR-compatible stereotactic frame can be designed and built with 3D printing, which will be attached to the MR scanner table and the patient's head to immobilize the head. To enable aberration correction, image-based registration can be used to align the pre-operative CT with the intra-operative MR images. The array and frame are shown in FIG. 3B.

3) The acoustic coupler can be a plastic membrane with one end sealed to the stereotactic frame and the other end sealed to the array via o-rings. The sealed coupler can have an inlet and outlet to circulate degassed water. Degassed water can directly interface the array and the skin of the patient's shaved head to avoid bubbles trapping on the head. Bubbles <1 mm will dissolve during pre-therapy imaging scans in the highly degassed water. To detect any larger bubbles, an MRI susceptibility weighted image (SWI) can be collected following initial localization, and a catheter can be placed through the inlet to remove them by suction.

4) A supporting structure can connect the positioner with the array and support the patient's neck, all in one portable frame. The entire tcMR-USgHt system can fit into the bore of a clinical MRI scanner with an inner diameter of 60 cm. The electrical driving system for the array and the water circulating system can be placed outside the MRI scanning room in an adjacent equipment room, with cables and hoses connected through wave guide penetrations.

The primary outcome will be the focal pressure through the skull after aberration correction. The transcranial histotripsy array of the present disclosure can achieve >30 MPa at locations ranging from the deep brain to <1 cm from the skull surface in >70% patients (based on the 50 CT scans+10 skulls and 10 cadavers).

Potential problems and alternative strategies: 1) Histotripsy uses extremely high pressure with high non-linearity, which cannot be fully replicated using the K-wave simulation. However, the f-number of transcranial array is very low (0.5), and the skull functions as a low-pass filter, both of which reduce the non-linear effect. Preliminary simulation results matched well with experimental results. 2) The PZT composite is ~20 times more expensive than PZT5. If the increased element number and packing density along with the PZT5 can achieve the goal, we will use the PZT5; otherwise PZT composite will be used.

Signal-to-noise ratio (SNR) and resolution constraints imposed by a lack of dedicated receive RF coils impaired imaging resolution and contrast. In one embodiment, a system can be configured to enhance MR image quality by integrating ultrasound-translucent RF receive coils into the histotripsy system developed above. Meanwhile, recent breakthroughs in driving electronics permit a single amplifier setup to both transmit histotripsy acoustic pulses and receive subsequent acoustic cavitation emission (ACE) signals. The present disclosure provides an ACE-based quantitative cavitation monitoring system to enable real-time treatment monitoring at a high frame rate (≥50 Hz) that is practical for histotripsy delivery as well as cavitation mapping of on-target and off-target locations including on the skull surface. This can be complemented by periodic updates from dMRI to provide tissue damage evaluation during histotripsy with both high spatial and temporal resolution. The color-encoded cavitation/damage map by ACE can be co-registered and superimposed over MR images to form integrated US and MR guidance. Together, this tcMR-USgHt system is configured to produce co-registered MRI and US treatment guidance and monitoring at high spatial and temporal resolution and at all intracranial locations.

The feasibility and accuracy of using MRI thermometry for pre-treatment targeting of histotripsy is described above. Histotripsy brain ablation can be visualized by dMRI ex vivo and in vivo, and AADC increases with increasing histotripsy dose applied (C.0.2). In vivo histotripsy brain ablation can also be visualized by T2- or T1-weighted MRI (C.0.2). However, use of a suboptimal RF receive coil reduces MR image resolution and SNR. Our collaborators at GE have shown that thin wire receive coils (AIR coils) placed between an ultrasound transducer and the subject will dramatically improve image SNR (13×) during FUS while minimizing ultrasound attention (<5%) for narrowband, thermal sonications 57 (FIGS. 9A-9B). The AIR coils can be used to enhance the MRI signals during histotripsy.

FIGS. 10A-10B show another example of a tcMR-USgHt system which includes a transducer 1000 having a focal point 1001, a water bath 1002, a FUS flex coil 1004 mounted on a stretchable fabric 1006 that covers the patient's face but still allows the patient to see and breathe, and feeding circuits 1008 for the FUS flex coil. Other components, such as the stereotactic frame and the robotic arm are not shown in this example.

The transmit-receive capable transcranial histotripsy array is configured to capture ACE and subsequently localized cavitation. Histotripsy can be delivered from the array through human skulls. Array elements are configured to operate in a transmit-receive mode and capture transcranial ACE signals. ACE signals can be detected through the human skulls. In some embodiments, cavitation can be mapped to within ~1.5 mm of its independently-measured position, at rates of up to 70 Hz. Cavitation maps in human brains can be overlaid on a T2-weighted MR image (FIG. 11A). Additionally, ACE-based cavitation maps can detect cavitation near or on the skull surface (FIGS. 11B-11C). As ACE signals vary from pulse-to-pulse while skull reflection signals remain the same from the same location, ACE and reflection signals can be separated.

FIG. 11A shows ACE-based localized cavitation map (with locations 1101—≥75% on-target; and locations 1103—<75% on-target), acquired transcranially using the transmit-receive capable 360-element histotripsy array, during treatment of planned cubic 1 cm³ volume in the human brain of a full cadaver, overlaid on a T2-weighted MR image. A cavitation map of representative cases of focal cavitation without off-target cavitation in FIG. 11B and skull outer surface cavitation without focal cavitation in FIG. 11C. The skull outer surface SoS is shown, with ultrasound applied from the bottom of the page.

Histotripsy-translucent surface coil arrays are provided that use flexible, thin-wire AIR coil designs. These coils have never previously been investigated for compatibility with high-bandwidth histotripsy acoustic pulses. A through-transmission setup can be used to measure acoustic attenuation and changes in sound speed and aberration induced by a single loop element of the coil array. Pressure thresholds for cavitation at the coil surface are measured. Finally, with the coil array in place, total acoustic pressure through a cadaver skull can be measured using a fiber optic hydrophone. The observed acoustic attenuation can be used to update the results above to map possible treatment locations within a human skull. Meanwhile, improvements in image SNR and resolution can be obtained by imaging the transducer and coil array in a 3T MRI scanner. SNR gain and suitability of the coil array for parallel imaging can assessed by computing the g-factor of the coil array using the pseudo-replica method. The measured SNR can be compared and contrasted of pre- and post-treatment images in phantoms and cadavers.

Monitoring of histotripsy-induced damage using MRI: Preliminary data suggests that dMRI may provide a quantitative and visual verification of the tissue damage generated by histotripsy. The dMRI approaches, as described in preliminary data, may first be optimized for the new array coils for spatial resolution, field of view, SNR, and parallel imaging acceleration. As previously mentioned, due to the small spatial and temporal footprint of individual histotripsy pulses, the direct observation of this effect is inefficient, so dMRI can be used to monitor in real-time the impact of tissue damage by calculating the apparent diffusion coefficient (ADC) every 6 second at a spatial resolution of 2×2×2 mm³, with a sliding window average, if necessary for SNR purposes. While there are numerous factors that can influence absolute ADC including tissue type (grey/white/CSF) and temperature (phantoms), quantitative changes in ADC (AADC) can indicate a degree of tissue damage. AADC and cavitation lifespan derived from ACE can be used to predict the level of histotripsy-induced damage.

US cavitation mapping-If cavitation is generated at the intended location, the arrival delays of the ACE signals at the array will equal the inverse of the transmit delays (including aberration correction) applied to steer the focus. This can be rapidly checked by phase-aligning the received signals against the inverse transmit delays and evaluating whether the timings of the detected ACE signals are temporally co-aligned. If they are, cavitation can be recorded as having occurred on-target, if not the acquired ACE signals will be processed using passive acoustic mapping (PAM) approaches. PAM localization utilizes coherent beamforming methods to identify the spatial location of an acoustic source by back-projecting acquired signals into the field, summing them together at each location, and identifying the position where the summed signal intensity is largest. PAM localization is typically carried out using signals acquired from diagnostic US imaging probes. Because the transducer elements in US imaging probes are tightly spatially packed, this often requires analyzing a large number of signals to accurately localize cavitation and the corresponding burdens associated with transferring said signals to a central processor for analysis is a primary contributor to limiting PAM localization rates to <50 Hz. As the histotripsy array elements are spatially well distributed, accurate PAM localization can be accomplished using a reduced number of signals, thus allowing higher localization rates to be achieved. Acquired ACE signals will be analyzed to identify optimal acquisition settings for achieving high PAM localization rates (≥50 Hz) while maintaining localization accuracy.

Skull surface cavitation mapping: As the acoustic background signals (e.g., reflections from the skull) do not vary pulse-to-pulse at the same location, cavitation generation near or on the skull surface can be detected by evaluating for deviations in the acoustic background in the signal regions nominally associated with skull reflections (FIG. 11C). If such background signal deviations are identified, PAM approaches will be used to localize any cavitation in the area. Aberration correction will be performed using a 2-step approach before PAM implementation.

The MRI and US guidance can be co-registered and integrated for the tcMR-USgHt system. In one example, a number of MRI fiducial markers (e.g., 4 marker) can be placed on the histotripsy array to co-register the location of the histotripsy array on MR images, and MR images to the ACE cavitation map. After pre-treatment targeting is performed by MR thermometry, the MR images can be exported to the computer controlling the histotripsy array. The target treatment volume can be outlined on pre-treatment MR images. During treatment, the processed ACE cavitation map with damage level color coded can be superimposed onto the co-registered MR anatomical images. After a predetermined time period (e.g., every 6 seconds or less), dMRI images can be exported, co-registered, and will also be overlaid with the ACE cavitation map. Cavitation lifespan and AADC can be calculated from ACE and dMRI, respectively, and used to predict the level of histotripsy damage. Post treatment, dMRI images exported to the computer with the lesion outlined, the transparent color cavitation map, and outline of the pre-treatment target volume will be compared with each other and correlated to the histology to calculate the treatment accuracy.

Quantitative evaluation of histotripsy-induced damage based on ACE and dMRI: Different levels of cellular damage can be created in ex vivo bovine brain tissue treated by the new transcranial histotripsy array through excised human skulls using different number of pulses. The damage level can be quantified with histology and compared to the cavitation lifespan extracted from the ACE signals and AADC from dMRI. In one experiment, a 3-cm diameter spherical ablation zone is treated with 1-50 pulses (in increments of 5) per location to generate different levels of induced damage at the treatment endpoints. 76 total samples are treated: 10 each in the ≤20 pulse cases, and 6 each in the 30-50 pulse cases—this distribution is chosen based on preliminary data. Following treatment, tissues can be fixed and stained for histology. Histological sections of the tissues, spanning the breadth of the treated regions (spacing between sections: 5 mm), will be acquired and evaluated for necrosis using H&E staining. A histological scoring system can be used to quantify induced damage (Grade 0-4; 0: no damage; 1:0-25% necrosis; 2:25-50% necrosis, 3:50-75% necrosis, 4: >75% necrosis), which will then be correlated with the cavitation lifespan and ΔADC. Analysis plan and sample size justification: The Pearson/Spearman correlation coefficient can be evaluated, along with a 95% confidence interval to quantify the association between each cavitation lifespan or ΔADC and histological damage outcomes. We hypothesize that the correlation is 0.8, with this sample size, the lower bound of the 95% two-sided confidence interval for the Pearson correlation is 0.71, which is higher than our acceptable correlation of 0.7.

Measures of success: the AIR coil array improves the MRI SNR by a factor of >13 and will enable parallel imaging with acceleration factors of 2-3 without observable aliasing artifacts. The AIR coil reduces focal pressure in the mid brain by <5% and near the inner skull surface by <10%. Cavitation can be localized and mapped in 3D at rates ≥50 Hz, accurate to within the diameters of the bubbles (≤3 mm) for focal and off-target cavitation (including skull surface). The cavitation lifespan (acquired at ≥50 Hz frame rate) and AADC (acquired at every 6 sec) are quantitatively correlated with histological damage outcomes with a Pearson/Spearman correlation coefficient of ≥0.8. The cavitation map will match well (≤3 mm difference) with pre-treatment target volume and post-treatment ablation zone on dMRI.

Potential problems & alternative strategies—In some cases, pathological tissues are located at or near a surface (e.g., near sulci or ventricles) in close contact with cerebrospinal fluid (CSF). Ablations in such regions could lead to perforations of meningeal layers which would allow the perfusion of CSF into the targeted region. CSF influx into the ablation volume would immediately change the local material properties which could impact ACE-based assessment of induced damage. If such effects are observed, dMRI may be used primarily for damage monitoring.

tcMR-USgHt workflow can be carried out using the new tcMR-USgHt system following six steps a human subject. 1) CT scan: Before the treatment, a CT scan of the skull or subject's head can be optionally obtained to use for acoustic simulation and aberration correction. 2) Subject Placement: The head or subject can be placed on the MRI bed, and the skull or the head can be connected to the histotripsy array via the stereotactic frame and acoustic coupler. Using the treatment planning software, the array geometric focus can be identified on the MR image using the fiducial markers on the array scaffold. The array can be moved by the positioner to place the array geometric focus within the target volume. 3) Aberration correction: aberration can be corrected using the 2-step correction approach based on prior CT scans of the patient's head co-registered to the MR images followed by ACE-based aberration correction. 4) Targeting: Pre-treatment targeting can be performed using the MRI thermometry to validate whether that the array focus is placed in a desired location (e.g., the center) of the target volume. The array can be moved by the positioner or electronic focal steering until the targeting is confirmed in the intended location. 5) Treatment delivery: Transcranial histotripsy can be delivered to the target volume, guided by real-time ACE-based cavitation mapping and damage monitoring and periodic dMRI. 6) Post-treatment confirmation: Post-treatment dMRI and correlative pathology of the treated brain can be obtained to assess the damage to the target and surrounding brain.

The treatment location envelop of the tcMR-USgHt system is characterized herein. First, CT scans can be obtained for the patient's skull and used to perform simulation as described above to estimate the treatment location envelop based on acoustic attenuation and pressure hotspots. The simulation result can be validated by creating a 3 cc (18 mm diameter) spherical lesion following the simulated envelop in regions near the top, anterior (frontal superficial), posterior (occipital superficial), and lateral (temporal superficial) of the skull, near the skull base midline (pons) and lateral (CP angle), and in the central region (caudate, putamen), resulting in a total of 6 lesions per head phantom. 1-cycle pulses can be used, p− of ~30 MPa, 200 Hz PRF (0.02% duty cycle), 50 pulses per focal location, 1.1 mm focal spacing, and electrical focal steering.

The steering strategy and parameters can be adjusted to maximize the treatment speed for different brain volumes while avoiding off-target cavitation and keeping the temperature increase in the skull and surrounding brain below 5° C. Lesions of 3 cc (18 mm diameter), 10 cc (27 mm diameter), and 20 cc (34 mm diameter) volumes will be created at three locations, 1) near the skullcap, 2) near the skull base, and 3) in the center of the brain. Three lesions can be generated per phantom for 3 different locations. Electrical focal steering can be used where >30 MPa can be reached, and beyond that the focus will be mechanically moved.

Treatment accuracy can be calculated by the differences of the center position and total volume between planned volume (based on pre-treatment MR thermometry), ACE cavitation map (collected during treatment), and the actual ablation volume (based on post-treatment dMRI).

MR thermometry can be used to measure the temperature increase in the brain.

As for additional details pertinent to the present invention, materials and manufacturing techniques may be employed as within the level of those with skill in the relevant art. The same may hold true with respect to method-based aspects of the invention in terms of additional acts commonly or logically employed. Also, it is contemplated that any optional feature of the inventive variations described may be set forth and claimed independently, or in combination with any one or more of the features described herein. Likewise, reference to a singular item, includes the possibility that there are plural of the same items present. More specifically, as used herein and in the appended claims, the singular forms “a,” “and,” “said,” and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation. Unless defined otherwise herein, all technical and scientific terms used herein have the same meaning as commonly understood by one of ordinary skill in the art to which this invention belongs. The breadth of the present invention is not to be limited by the subject specification, but rather only by the plain meaning of the claim terms employed.

1. An MRI compatible histotripsy system, comprising:
a transducer array comprising a plurality of transducer elements;
transmission circuitry coupled to the transducer array and configured to transmit histotripsy pulses into a target tissue;
a MRI system configured to generate MR images of a target tissue of the subject; and
one or more coils placed between the MRI system and a subject, the one or more coils being configured to receive MRI signals from the MRI system to improve an image signal-to-noise-ratio (SNR) of the MR images of the MRI system.

2. The system of claim 1, wherein the one or more coils are ultrasound translucent.

3. The system of claim 1, wherein the transducer array comprises a transcranial transducer array and the one or more coils are positioned on a stretchable fabric.

4. The system of claim 1, wherein the transducer array is configured to deliver histotripsy pulses into a focal location to generate less than 2 deg C of heating at the focal location.

5. The system of claim 4, wherein the histotripsy pulses are modified from conventional histotripsy pulses to generate the heating.

6. The system of claim 1, wherein the MRI system is configured to generate MR thermometry images of the heating at the focal location.

7. The system of claim 1, further comprising one or more processors configured to determine if cavitation is being generated at a focus of the transducer array based on the MR thermometry images.

8. The system of claim 7, wherein the one or more processors are configured to measure a distance between a center of a heating map in the MR thermometry images and the focus.

9. The system of claim 1, further comprising identifying an apparent diffusion coefficient (ADC) in the MR images to identify tissue changes in the targe tissue.

10. The system of claim 1, wherein the transducer array is MR compatible.

11. A transcranial ultrasound treatment system, comprising:
a magnetic resonance (MR)-compatible histotripsy therapy array;
a MR-compatible mechanical positioner with translation and rotation mobility configured to move the histotripsy therapy array;
a stereotactic frame configured to rigidly fix a patient's head and facilitate aberration correction;
an acoustic coupler acoustically coupled to the histotripsy therapy array to provide for ultrasound transmission from the histotripsy therapy array to the patient's head; and
a support structure configured to connect the MR-conditional mechanical positioner with the histotripsy therapy array and to support the patient's neck.

12. The system of claim 11, further comprising a magnetic resonance imaging (MRI) system configured to generate MR-images of a target tissue.

13. The system of claim 12, further comprising one or more coils placed between the MRI system and the patient, the one or more coils being configured to receive MRI signals from the MRI system to improve an image signal-to-noise-ratio (SNR) of the MR images of the MRI system.

14. The system of claim 13, wherein the one or more coils are ultrasound translucent.

15. The system of claim 12, wherein the transducer array comprises a transcranial transducer array and the one or more coils are positioned on a stretchable fabric.

16. The system of claim 11, wherein the transducer array is configured to deliver histotripsy pulses into a focal location to generate less than 2 deg C. of heating at the focal location.

17. The system of claim 16, wherein the histotripsy pulses are modified from conventional histotripsy pulses to generate the heating.

18. The system of claim 12, wherein the MRI system is configured to generate MR thermometry images of the heating at the focal location.

19. The system of claim 12, further comprising one or more processors configured to determine if cavitation is being generated at a focus of the transducer array based on the MR thermometry images.

20. The system of claim 19, wherein the one or more processors are configured to measure a distance between a center of a heating map in the MR thermometry images and the focus.

21. The system of claim 12, further comprising identifying an apparent diffusion coefficient (ADC) in the MR images to identify tissue changes in the targe tissue.

22.-31. (canceled)
